SEQUENCE LISTING (86 sequences)

SEQ ID NO: 1 - protein, 15 aa, Hepatitis C virus
IPVENLETTMRSPVF

SEQ ID NO: 2 - protein, 15 aa, Hepatitis C virus
NLETTMRSPVFTDNS

SEQ ID NO: 3 - protein, 15 aa, Hepatitis C virus
PPAVPQSFQVAHLHA

SEQ ID NO: 4 - protein, 15 aa, Hepatitis C virus
PQSFQVAHLHAPTGS

SEQ ID NO: 5 - protein, 15 aa, Hepatitis C virus
FQVAHLHAPTGSGKS

SEQ ID NO: 6 - protein, 4 aa, Hepatitis C virus
DECH

SEQ ID NO: 7 - protein, 191 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASA

SEQ ID NO: 8 - protein, 191 aa, Hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASA

SEQ ID NO: 9 - protein, 192 aa, Hepatitis C virus
YQVRNSSGLYHVTNDCPNSSVVYEAADAILHTPGCVPCVREGNASRCWVAVTPTVATRDGKLPTTQLRRHIDLLVGSATLCSALYVGDLCGSVFLVGQLFTFSPRHHWTTQDCNCSIYPGHITGHRMAWNMMMNWSPTAALVVAQLLRIPQAIMDMIAGAHWGVLAGIKYFSMVGNWAKVLVVLLLFAGVDA

SEQ ID NO: 10 - protein, 363 aa, Hepatitis C virus
ETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKYPEATYSRCGSGPRITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 11 - protein, 63 aa, Hepatitis C virus
ALENLVILNAASLAGTHGLVSFLVFFCFAWYLKGRWVPGAVYALYGMWPLLLLLLALPQRAYA

SEQ ID NO: 12 - protein, 217 aa, Hepatitis C virus
LDTEVAASCGGVVLVGLMALTLSPYYKRYISWCMWWLQYFLTRVEAQLHVWVPPLNVRGGRDAVILLTCVVHPALVFDITKLLLAIFGPLWILQASLLKVPYFVRVQGLLRICALARKIAGGHYVQMAIIKLGALTGTCVYNHLAPLRDWAHNGLRDLAVAVEPVVFSRMETKLITWGADTAACGDIINGLPVSARRGQEILLGPADGMVSKGWRLL

SEQ ID NO: 13 - protein, 631 aa, Hepatitis C virus
APITAYAQQTRGLLGCIITSLTGRDKNQVEGEVQIVSTAAQTFLATCINGVCWTVYHGAGTRTIASSKGPVIQMYTNVDQDLVGWPAPQGARSLTPCTCGSSDLYLVTRHADVIPVRRRGDSRGSLLSPRPISYLKGSSGGPLLCPAGHAVGIFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPVTKYIMTCMSADLEVVT

SEQ ID NO: 14 - protein, 631 aa, Hepatitis C virus
APITAYSQQTRGLLGCIITSLTGRDKNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIETIKGGRHLIFCHSKKKCDELAAKLSGLGLNAVAYYRGLDVSVIPTSGDVVVVATDALMTGFTGDFDSVIDCNTCVTQTVDFSLDPTFTIETTTVPQDAVSRSQRRGRTGRGRRGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKYIMACMSADLEVVT

SEQ ID NO: 15 - protein, 54 aa, Hepatitis C virus
STWVLVGGVLAALAAYCLSTGCVVIVGRIVLSGKPAIIPDREVLYQEFDEMEEC

SEQ ID NO: 16 - protein, 261 aa, Hepatitis C virus
SQHLPYIEQGMMLAEQFKQKALGLLQTASRHAEVITPAVQTNWQKLEVFWAKHMWNFISGIQYLAGLSTLPGNPAIASLMAFTAAVTSPLTTGQTLLFNILGGWVAAQLAAPGAATAFVGAGLAGAALDSVGLGKVLVDILAGYGAGVAGALVAFKIMSGEVPSTEDLVNLLPAILSPGALAVGVVFASILRRRVGPGEGAVQWMNRLIAFASRGNHVSPTHYVPESDAAARVTAILSSLTVTQLLRRLHQWISSECTTPC

SEQ ID NO: 17 - protein, 448 aa, Hepatitis C virus
SGSWLRDIWDWICEVLSDFKTWLKAKLMPQLPGIPFVSCQRGYRGVWRGDGIMHTRCHCGAEITGHVKNGTMRIVGPRTCKNMWSGTFFINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFAPALPVWARPDYNPLLVETWKKPDYEPPVVHGCPLPPPRSPPVPPPRKKRTVVLTESTLPTALAELATKSFGSSSTSGITGDNTTTSSEPAPSGCPPDSDVESYSSMPPLEGEPGDPDLSDGSWSTVSSGADTEDVVCC

SEQ ID NO: 18 - protein, 591 aa, Hepatitis C virus
SMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVNHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKAQAACRAAGLRDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGQLDLSGWFTAGYSGGDIYHSVSRARPRWFWFCLLLLAAGVGIYLLPNR

SEQ ID NO: 19 - protein, 591 aa, Hepatitis C virus
SMSYTWTGALITPCAAEESKLPINALSNSLLRHHNMVYATTSRSASQRQKKVTFDRLQVLDDHYRDVLKEMKAKASTVKAKLLSVEEACKLTPPHSAKSKFGYGAKDVRNLSSKAVNHIRSVWKDLLEDTETPIDTTIMAKNEVFCVQPEKGGRKPARLIVFPDLGVRVCEKMALYDVVSTLPQAVMGSSYGFQYSPGQRVEFLVNAWKSKKNPMGFAYDTRCFDSTVTENDIRVEESIYQCCDLAPEARQAIRSLTERLYIGGPLTNSKGQNCGYRRCRASGVLTTSCGNTLTCYLKASAACRAAKLQDCTMLVCGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIQRLHGLSAFSLHSYSPGEINRVASCLRKLGVPPLRVWRHRARSVRAKLLSQGGRAATCGKYLFNWAVRTKLKLTPIPAASQLDLSGWFVAGYSGGDIYHSLSRARPRWFMLCLLLLSVGVGIYLLPNR

SEQ ID NO: 20 - protein, 631 aa, Hepatitis C virus
APITAYAQQTRGLLGCIITSLTGRDKNQVEGEVQIVSTAAQTFLATCINGVCWTVYHGAGTRTIASSKGPVIQMYTNVDQDLVGWPAPQGARSLTPCTCGSSDLYLVTRHADVIPVRRRGDSRGSLLSPRPISYLKGSSGGPLLCPAGHAVGIFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGIDPNIRTGVRTITTGSPITYSTYGKFLADGGCSGGAYDIIICAASHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPVTKYIMTCMSADLEVVT

SEQ ID NO: 21 - protein, 631 aa, Hepatitis C virus
APITAYSQQTRGLLGCIITSLTGRDKNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICAASHSTDSTTILGIGTVLDQAETAGARLVVLATATPPGSVTVPHPNIEEVALSNTGEIPFYGKAIPIETIKGGRHLIFCHSKKKCDELAAKLSGLGLNAVAYYRGLDVSVIPTSGDVVVVATDALMTGFTGDFDSVIDCNTCVTQTVDFSLDPTFTIETTTVPQDAVSRSQRRGRTGRGRRGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKYIMACMSADLEVVT

SEQ ID NO: 22 - protein, 591 aa, Hepatitis C virus
SMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVNHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKAQAACRAAGLRDCTMLVCGNLLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGQLDLSGWFTAGYSGGDIYHSVSRARPRWFWFCLLLLAAGVGIYLLPNR

SEQ ID NO: 23 - protein, 591 aa, Hepatitis C virus
SMSYTWTGALITPCAAEESKLPINALSNSLLRHHNMVYATTSRSASQRQKKVTFDRLQVLDDHYRDVLKEMKAKASTVKAKLLSVEEACKLTPPHSAKSKFGYGAKDVRNLSSKAVNHIRSVWKDLLEDTETPIDTTIMAKNEVFCVQPEKGGRKPARLIVFPDLGVRVCEKMALYDVVSTLPQAVMGSSYGFQYSPGQRVEFLVNAWKSKKNPMGFAYDTRCFDSTVTENDIRVEESIYQCCDLAPEARQAIRSLTERLYIGGPLTNSKGQNCGYRRCRASGVLTTSCGNTLTCYLKASAACRAAKLQDCTMLVCGNLLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIQRLHGLSAFSLHSYSPGEINRVASCLRKLGVPPLRVWRHRARSVRAKLLSQGGRAATCGKYLFNWAVRTKLKLTPIPAASQLDLSGWFVAGYSGGDIYHSLSRARPRWFMLCLLLLSVGVGIYLLPNR

SEQ ID NO: 24 - protein, 18 aa, Artificial Sequence
  Description of Artificial Sequence Synthetic peptide
ATDSEDSSLNTDEWEEEK

SEQ ID NO: 25 - protein, 8 aa, Artificial Sequence
  Description of Artificial Sequence Synthetic peptide
SIINFEKL

SEQ ID NO: 26 - protein, 237 aa, Listeria monocytogenes
MNAQAEEFKKYLETNGIKPKQFHKKELIFNQWDPQEYCIFLYDGITKLTSISENGTIMNLQYYKGAFVIMSGFIDTETSVGYYNLEVISEQATAYVIKINELKELLSKNLTHFFYVFQTLQKQVSYSLAKFNDFSINGKLGSICGQLLILTYVYGKETPDGIKITLDNLTMQELGYSSGIAHSSAVSRIISKLKQEKVIVYKNSCFYVQNLDYLKRYAPKLDEWFYLACPATWGKLN

SEQ ID NO: 27 - protein, 237 aa, Listeria monocytogenes
MNAQAEEFKKYLETNGIKPKQFHKKELIFNQWDPQEYCIFLYDGITKLTSISENGTIMNLQYYKGAFVIMSGFIDTETSVGYYNLEVISEQATAYVIKINELKELLSKNLTHFFYVFQTLQKQVSYSLAKFNDFSINGKLGSICGQLLILTYVYSKETPDGIKITLDNLTMQELGYSSGIAHSSAVSRIISKLKQEKVIVYKNSCFYVQNLDYLKRYAPKLDEWFYLACPATWGKLN

SEQ ID NO: 28 - protein, 5 aa, Listeria monocytogenes
TEAKD

SEQ ID NO: 29 - protein, 5 aa, Lactococcus lactis
VYADT

SEQ ID NO: 30 - protein, 5 aa, Bacillus anthracis
IQAEV

SEQ ID NO: 31 - protein, 5 aa, Listeria monocytogenes
ASAST

SEQ ID NO: 32 - protein, 5 aa, Listeria monocytogenes
VGAFG

SEQ ID NO: 33 - protein, 5 aa, Bacillus anthracis
AFAED

SEQ ID NO: 34 - protein, 5 aa, Staphylococcus aureus
VQAAE

SEQ ID NO: 35 - protein, 5 aa, Listeria monocytogenes
DKALT

SEQ ID NO: 36 - protein, 5 aa, Bacillus subtillis
VGAFG

SEQ ID NO: 37 - protein, 100 aa, Listeria monocytogenes
VGLNRFMRAMMVVFITANCITINPDIIFAATDSEDSSLNTDEWEEEKTEEQPSEVNTGPRYETAREVSSRDIEELEKSNKVKNTNKADLIAMLKAKAEKG

SEQ ID NO: 38 - protein, 100 aa, Listeria monocytogenes
MGLNRFMRAMMVVFITANCITINPDIIFAATDSEDSSLNTDEWEEEKTEEQPSEVNTGPRYETAREVSSRDIEELEKSNKVKNTNKADLIAMLKAKAEKG

SEQ ID NO: 39 - protein, 102 aa, Artificial Sequence
  Description of Artificial Sequence Synthetic polypeptide
VGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGGS

SEQ ID NO: 40 - protein, 102 aa, Artificial Sequence
  Description of Artificial Sequence Synthetic polypeptide
MGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGGS

SEQ ID NO: 41 - protein, 29 aa, Listeria monocytogenes
MGLNRFMRAMMVVFITANCITINPDIIFA

SEQ ID NO: 42 - protein, 20 aa, Hepatitis C virus
LETTMRSPVFTDNSSPPVVP

SEQ ID NO: 43 - protein, 15 aa, Hepatitis C virus
SPVFTDNSSPPAVPQ

SEQ ID NO: 44 - protein, 15 aa, Hepatitis C virus
VPQSFQVAHLHAPTG

SEQ ID NO: 45 - protein, 15 aa, Hepatitis C virus
FQVAHLHAPTGSGKS

SEQ ID NO: 46 - protein, 15 aa, Hepatitis C virus
KVPAAYAAQGYKVLV

SEQ ID NO: 47 - protein, 20 aa, Hepatitis C virus
PAAYAAQGYKVLVLNPSVAA

SEQ ID NO: 48 - protein, 15 aa, Hepatitis C virus
AAKGYKVLVLNPSVA

SEQ ID NO: 49 - protein, 10 aa, Hepatitis C virus
VLVLNPSVAA

SEQ ID NO: 50 - protein, 15 aa, Hepatitis C virus
AQGYKVLVLNPSVAA

SEQ ID NO: 51 - protein, 14 aa, Hepatitis C virus
QGYKVLVLNPSVAA

SEQ ID NO: 52 - protein, 14 aa, Hepatitis C virus
GYKVLVLNPSVAAT

SEQ ID NO: 53 - protein, 20 aa, Hepatitis C virus
GYKVLVLNPSVAATLGFGAY

SEQ ID NO: 54 - protein, 20 aa, Hepatitis C virus
GVRTITTGSPITYSTYGKFL

SEQ ID NO: 55 - protein, 20 aa, Hepatitis C virus
ITYSTYGKFLADGGCSGGAY

SEQ ID NO: 56 - protein, 18 aa, Hepatitis C virus
LADAGCSGGAYDIIICDE

SEQ ID NO: 57 - protein, 15 aa, Hepatitis C virus
GGAYDIIICDECHST

SEQ ID NO: 58 - protein, 15 aa, Hepatitis C virus
DIIICDECHSTDATS

SEQ ID NO: 59 - protein, 20 aa, Hepatitis C virus
TDATSILGIGTVLDQAETAG

SEQ ID NO: 60 - protein, 15 aa, Hepatitis C virus
ATSILGIGTVLDQAE

SEQ ID NO: 61 - protein, 18 aa, Hepatitis C virus
VIKGGRHLIFCHSKKKCD

SEQ ID NO: 62 - protein, 11 aa, Hepatitis C virus
GRHLIFCHSKR

SEQ ID NO: 63 - protein, 15 aa, Hepatitis C virus
KCDELAAKLVALGIN

SEQ ID NO: 64 - protein, 20 aa, Hepatitis C virus
GINAVAYYRGLDVSVIPTSG

SEQ ID NO: 65 - protein, 18 aa, Hepatitis C virus
IPTNGDVVVVSTDALMTG

SEQ ID NO: 66 - protein, 15 aa, Hepatitis C virus
ALMTGYTGDFDSVID

SEQ ID NO: 67 - protein, 18 aa, Hepatitis C virus
DFDSVIDCNTCVTQTVDF

SEQ ID NO: 68 - protein, 20 aa, Hepatitis C virus
SVIDCNTCVTQTVDFSLDPT

SEQ ID NO: 69 - protein, 18 aa, Hepatitis C virus
CNTCVTQTVDFSLDPTFT

SEQ ID NO: 70 - protein, 15 aa, Hepatitis C virus
NTCVTQTVDFSLDPT

SEQ ID NO: 71 - protein, 18 aa, Hepatitis C virus
PTFTIETTTLPQDAVSRT

SEQ ID NO: 72 - protein, 15 aa, Hepatitis C virus
TQTVDFSLDPTFTIE

SEQ ID NO: 73 - protein, 15 aa, Hepatitis C virus
EQYVDFSLDPTFSIE

SEQ ID NO: 74 - protein, 20 aa, Hepatitis C virus
LRHHNLVYSTTSRSACQRQK

SEQ ID NO: 75 - protein, 20 aa, Hepatitis C virus
KVTFDRLQVLDSHYQDVLKE

SEQ ID NO: 76 - protein, 20 aa, Hepatitis C virus
SVWKDLLEDNVTPIDTTIMA

SEQ ID NO: 77 - protein, 20 aa, Hepatitis C virus
KGGRKPARLIVFPDLGVRVC

SEQ ID NO: 78 - protein, 18 aa, Hepatitis C virus
KPARLIVFPDLGVRVCEK

SEQ ID NO: 79 - protein, 20 aa, Hepatitis C virus
KLPLAVMGSSYGFQYSPGQR

SEQ ID NO: 80 - protein, 20 aa, Hepatitis C virus
VEFLVQAWKSKKTPMGFSYD

SEQ ID NO: 81 - protein, 20 aa, Hepatitis C virus
SDIRTEEAIYQCCDLDPQAR

SEQ ID NO: 82 - protein, 20 aa, Hepatitis C virus
QCCDLDPQARVAIKSLTERL

SEQ ID NO: 83 - protein, 12 aa, Hepatitis C virus
GYRRCRASGVLT

SEQ ID NO: 84 - protein, 4 aa, Hepatitis C virus
AASH

SEQ ID NO: 85 - protein, 10 aa, Hepatitis C virus
KLVALGINAV

SEQ ID NO: 86 - protein, 9 aa, Hepatitis C virus
ALYDVVSKL

(19) United States
(12) Patent Application Publication
(10) Pub. No.: US 2012/0100170 A1
(43) Pub. Date: Apr. 26, 2012

(54) COMPOSITIONS AND METHODS FOR THE TREATMENT OF HEPATITIS C

(76) Inventors: Peter M. Lauer, Albany, CA (US); Thomas W. Dubensky, JR., Seattle, WA (US)

(21) Appl. No.: 13/055,675

(22) PCT Filed: Jul. 23, 2009
(86) PCT No.: PCT/US09/51596

§ 371 Date: Sep. 21, 2011

Related U.S. Application Data
(60) Provisional application No. 61/083,487, filed on Jul. 24, 2008.

(51) Int. Cl. A61K 39/29; A61P 31/14; A61P 37/04

(52) U.S. Cl. 424/189.1; 424/200.1

(57) ABSTRACT

The present invention provides compositions and methods for delivery of one or more hepatitis C virus (HCV) antigens using a bacterium recombinantly encoding and expressing such antigens. In certain embodiments, the bacterial platform comprises the use of attenuated and killed but metabolically active forms of Listeria monocytogenes.

### STATEMENT REGARDING FEDERALLY SPONSORED RESEARCH OR DEVELOPMENT

This invention was made, in part, with U.S. government support under Grant No. 1 U01 AI070834-01 awarded by The National Institutes of Health. The government has certain rights in the invention.

### FIELD OF THE INVENTION

The present invention relates generally to treatment of subjects suffering from or at risk of suffering from hepatitis C infection. More particularly, the present invention relates to compositions and methods for delivery of one or more hepatitis C virus (HCV) antigens using a bacterium recombinantly encoding and expressing such antigens.

### BACKGROUND OF THE INVENTION

The following discussion of the background of the invention is merely provided to aid the reader in understanding the invention and is not admitted to describe or constitute prior art to the present invention.

Hepatitis C is a major cause of morbidity and mortality worldwide. An estimated 170 million individuals are infected with HCV worldwide, with nearly 4 million people chronically infected in the U.S. and up to 9 million people chronically infected in Europe. Acute disease may lead to recovery, fulminant hepatitis, relapsing hepatitis with intervening periods of normal liver function, inapparent chronic infection, chronic active hepatitis and cirrhosis. Of those exposed to HCV, 80% become chronically infected, and at least 30% of carriers develop chronic liver disease, including cirrhosis and hepatocellular carcinoma.

The current standard of care for patients in developed countries with chronic HCV infection, interferon-α (IFN-α) and ribavirin, has demonstrated differential effectiveness amongst the most prevalent HCV genotypes 1-3. While effective in approximately 80% of patients with HCV genotypes 2 and 3, only about 50% of patients chronically infected with HCV genotype 1 exhibit a sustained viral response following treatment with IFN-α/ribavirin. Between 70-80% of the chronic HCV infections in the U.S. are genotype 1. In addition, the toxicity and tolerability profiles of IFN-α and ribavirin limit their use in HCV treatment.

Although a number of investigational agents are in clinical development, including immune-based therapies and small molecules targeting the function of specific HCV gene products such as the viral proteinase (NS3) and the viral polymerase (NS5B). For example, IC41 is a synthetic peptide vaccine containing 7 relevant hepatitis C virus (HCV) T-cell epitopes and the T helper cell (Th)1/Tc1 adjuvant poly-L-arginine. IC41 reportedly induced HCV-specific interferon (IFN)-gamma-secreting CD4+ and CD8+ T cells in healthy volunteers. Recombinant HCV NS3 and NS5B proteins together with an adjuvant mixture comprising M-ISA720 and CpG dinucleotides also reportedly induced CD4(+) and CD8(+) T cell responses. None of these approaches have yet to establish superior effectiveness and tolerability over the current standard of care in the chronic HCV setting.

### SUMMARY OF THE INVENTION

The present invention provides compositions and methods for delivery of one or more hepatitis C virus (HCV) antigens using a bacterium recombinantly encoding and expressing such antigens.

In a first aspect of the invention, the invention relates to methods of inducing a T-cell response to hepatitis C virus (HCV) in a subject. These method comprise administering to a subject a composition comprising a bacterium which expresses one or more immunogenic HCV antigen polypeptides, the amino acid sequence of which comprise

(i) one or more full length HCV proteins selected from the group consisting of core, E1, E2, p7, NS2, NS3, NS4a, NS4b, NS5a, and NS5b;
(ii) one or more immunogenic amino acid sequences derived from one or more full length HCV proteins from (i); or
a combination of one or more full length HCV proteins of (i) and one or more amino acid sequences of (ii).

As described herein, such methods can stimulate an antigen-specific T cell (CD4+ and/or CD5+) response in said subject to the recombinantly expressed immunogenic HCV antigen polypeptides. Preferably, when delivered to the subject, the compositions of the present invention induce an increase in the serum concentration of one or more, and preferably each of, proteins selected from the group consisting of IL-12p70, IFN-γ, IL-6, TNF α, and MCP-1 at 24 hours following said delivery; and induce a CD4+ and/or CD8+ antigen-specific T cell response against one or more of said immunogenic HCV antigen polypeptide(s) expressed by the bacterium.

In a related aspect of the invention, the invention relates to compositions useful for inducing a T-cell response to hepatitis C virus (HCV) in a subject. Such compositions comprise a bacterium which comprises a nucleic acid molecule, the sequence of which encodes one or more immunogenic HCV antigen polypeptides, the amino acid sequence of which comprise

(i) one or more full length HCV proteins selected from the group consisting of core, E1, E2, p7, NS2, NS3, NS4a, NS4b, NS5a, and NS5b;
(ii) one or more immunogenic amino acid sequences derived from one or more full length HCV proteins from (i); or
a combination of one or more full length HCV proteins of (i) and one or more amino acid sequences of (ii).

And in another related aspect, the invention relates to a isolated nucleic acid molecule, the sequence of which encodes one or more immunogenic HCV antigen polypeptides, the amino acid sequence of which comprise

(i) one or more full length HCV proteins selected from the group consisting of core, E1, E2, p7, NS2, NS3, NS4a, NS4b, NS5a, and NS5b;
(ii) one or more immunogenic amino acid sequences derived from one or more full length HCV proteins from (i); or
a combination of one or more full length HCV proteins of (i) and one or more amino acid sequences of (ii).

Methods for selecting appropriate immunogenic HCV antigen polypeptide sequences are described in detail hereinafter, and exemplary immunogenic HCV antigen polypeptide sequences are provided. Selection methods can comprise the selection of one or more contiguous HCV amino acid sequences having no region of hydrophobicity that exceeds the peak hydrophobicity of Listeria ActA-N100; the selection of one or more contiguous HCV amino acid sequences predicted to encode one or more MHC class I epitopes; and/or the selection of one or more contiguous HCV amino acid sequences predicted to encode one or more MHC class I epitopes. The ability of such polypeptides to generate a CD4+ and/or CD8+ T cell response may be confirmed by a variety of methods described in detail herein and that are well known in the art.

In certain embodiments, the immunogenic HCV antigen polypeptide(s) comprise one or more amino acid sequences which are independently selected from the group consisting of full length core, E1, E2, p7, NS2, NS3, NS4a, NS4b, NS5a, and NS5b; an amino acid sequence having at least 90% sequence identity to such full length HCV antigens; a fragment of core, E1, E2, p7, NS2, NS3, NS4a, NS4b, NS5a, and NS5b; and an amino acid sequence having at least 90% sequence identity to such fragments.

In preferred embodiments, the immunogenic HCV antigen polypeptide(s) comprise one or more amino acid sequences selected from the group consisting of full length NS3, full length NS5b, an amino acid sequence derived from NS3, and an amino acid sequence derived from NS5b. In certain embodiments, the derived amino acid sequence(s) may be independently selected from the group consisting of an amino acid sequence comprising at least 100 contiguous residues from NS3; an amino acid sequence comprising at least 100 contiguous residues from NS5b; an amino acid sequence having at least 90% sequence identity to at least 100 contiguous residues from NS3, and an amino acid sequence having at least 90% sequence identity to at least 100 contiguous residues from NS5b.

Numerous HCV isolates which may serve as the source material for the foregoing amino acid sequences are known in the art. In preferred embodiments, the core, E1, E2, p7, NS2, NS3, NS4a, NS4b, NS5a, and/or NS5b amino acid sequences are consensus sequences, preferably from a genotype 1 HCV consensus sequence, and most preferably from a genotype 1a or 1b consensus sequence. Exemplary consensus sequences are provided hereinafter. The sequence of a protein may be modified by one or more insertions, deletions, and/or substitutions

Particularly preferred HCV antigen polypeptide(s) comprise one or more, and preferably each of, amino acid sequences selected from the group consisting of SEQ ID NOS: 1, 2, 3, 4, 5, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, and 83.

A number of bacterial species have been developed for use as vaccines and can be used as a vaccine platform in present invention, including, but not limited to, Shigella flexneri, Escherichia coli, Listeria monocytogenes, Yersinia enterocolitica, Salmonella typhimurium, Salmonella typhi or mycobacterium species. This list is not meant to be limiting. The present invention contemplates the use of attenuated, commensal, and/or killed but metabolically active bacterial strains as vaccine platforms. In preferred embodiments the bacterium is Listeria monocytogenes comprising a nucleic acid sequence encoding for expression by the bacterium one or more immunogenic HCV antigen polypeptides of the invention. This nucleic acid is most preferably integrated into the genome of the bacterium. Attenuated and killed but metabolically active forms of Listeria monocytogenes are particularly preferred, and Listeria monocytogenes harboring an attenuating mutation in actA and/or inlB is described hereinafter in preferred embodiments.

The vaccine compositions described herein can be administered to a host, either alone or in combination with a pharmaceutically acceptable excipient, in an amount sufficient to induce an appropriate immune response to HCV infection. Preferred conditions selected to induce a T cell response in a subject comprise administering the vaccine platform intravenously to a subject; however, administration may be oral, intravenous, subcutaneous, dermal, intradermal, intramuscular, mucosal, parenteral, intraorgan, intralesional, intranasal, inhalation, intraocular, intravascular, intranodal, by scarification, rectal, intraperitoneal, or any one or combination of a variety of well-known routes of administration.

In certain preferred embodiments, after the subject has been administered an effective dose of a vaccine containing the immunogenic HCV antigen polypeptides to prime the immune response, a second vaccine is administered. This is referred to in the art as a “prime-boost” regimen. In such a regimen, the compositions and methods of the present invention may be used as the “prime” delivery, as the “boost” delivery, or as both a “prime” and a “boost.” Examples of such regimens are described hereinafter.

A preferred Listeria monocytogenes for use in the present invention comprises a mutation in the prfA gene which locks the expressed prfA transcription factor into a constitutively active state. For example, a PrfA* mutant (G155S) has been shown to enhance functional cellular immunity following a prime-boost intravenous or intramuscular immunization regimen.

In certain embodiments, the immunogenic HCV antigen polypeptide(s) are expressed as one or more fusion proteins comprising an in frame secretory signal sequence, thereby resulting in secretion of soluble HCV antigen polypeptide(s) by the bacterium. Numerous exemplary signal sequences are known in the art for use in bacterial expression systems. In the case where the bacterium is Listeria monocytogenes, it is preferred that the secretory signal sequence is a Listeria monocytogenes signal sequence, most preferably the ActA signal sequence. Additional ActA or othet linker amino acids may also be expressed fused to the immunogenic HCV antigen polypeptide(s). In preferred embodiments, one or more immunogenic HCV antigen polypeptide(s) are expressed as fusion protein(s) comprising an in frame ActA-N100 sequence (e.g., selected from the group consisting of SEQ ID NO: 37, SEQ ID NO: 38, SEQ ID NO: 39, SEQ ID NO: 40) or an amino acid sequence having at least 90% sequence identity to said ActA-N100 sequence.

In preferred embodiments, the vaccine composition comprises a Listeria monocytogenes expressing a fusion protein comprising:

(a) an ActA-N100 sequence selected from the group consisting of SEQ ID NO: 37, SEQ ID NO: 38, SEQ ID NO: 39, SEQ ID NO: 40 or an amino acid sequence having at least 90% sequence identity to this ActA-N100 sequence;
(b) an amino acid sequence comprising at least 100 contiguous residues from NS3 or an amino acid sequence having at least 90% sequence identity to an amino acid sequence comprising at least 100 contiguous residues from NS3; and
(c) an amino acid sequence comprising at least 100 contiguous residues from NS5b or an amino acid sequence having at least 90% sequence identity to an amino acid sequence comprising at least 100 contiguous residues from NS5b;
wherein the fusion protein is expressed from a nucleic acid sequence operably linked to a Listeria monocytogenes ActA promoter. In particularly preferred embodiments, the amino acid sequences of (c) comprise amino acids 1-342 of NS5b (and preferably comprising amino acids 1-342 of SEQ ID NO: 18 or SEQ ID NO: 19) or a mutated derivative thereof, wherein said mutation inactivates the RNA polymerase activity of NS5b (and preferably the mutation depicted in SEQ ID NO: 22 or SEQ ID NO: 23); and the amino acid sequences of (b) comprise amino acids 172-484 of NS3 (and preferably comprising amino acids 172-484 of SEQ ID NO: 13 or SEQ ID NO: 14) or a mutated derivative thereof, wherein said mutation inactivates the helicase activity of NS3 (and preferably the mutation depicted in SEQ ID NO: 20 or SEQ ID NO: 21).

In the case of expression from a Listeria monocytogenes bacterium, in certain embodiments the nucleic acid sequences encoding the HCV antigen polypeptide(s) are codon optimized for expression by Listeria monocytogenes. As described hereinafter, different organisms often display “codon bias”; that is, the degree to which a given codon encoding a particular amino acid appears in the genetic code varies significantly between organisms. In general, the more rare codons that a gene contains, the less likely it is that the heterologous protein will be expressed at a reasonable level within that specific host system. These levels become even lower if the rare codons appear in clusters or in the N-terminal portion of the protein. Replacing rare codons with others that more closely reflect the host system's codon bias without modifying the amino acid sequence can increase the levels of functional protein expression. Methods for codon optimization are described hereinafter.

The methods and compositions of the present invention may find use as both a prophylactic or as a therapeutic HCV vaccine. In preferred embodiments, a subject is selected to receive the compositions of the present invention based on a previously diagnosed chronic HCV infection.

It is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

The present invention and the various features and advantageous details thereof are explained more fully with reference to the non-limiting embodiments that are illustrated in the accompanying drawings and detailed in the following description. It should be noted that the features illustrated in the drawings are not necessarily drawn to scale. Descriptions of well-known components and processing techniques are omitted so as to not unnecessarily obscure the present invention. The examples used herein are intended merely to facilitate an understanding of ways in which the invention may be practiced and to further enable those of skill in the art to practice the invention. Accordingly, the examples should not be construed as limiting the scope of the invention. In the drawings, like reference numerals designate corresponding parts throughout the several views.

FIG. 1 depicts a schematic of the derivation of the L. monocytogenes vaccine strain ANZ-521

FIG. 2 depicts a schematic of the HCV NS5B-NS3 antigen expression cassette inserted at the inlB Locus of Listeria monocytogenes ANZ 100.

FIG. 3 depicts various constructs used in the construction of Listeria monocytogenes ANZ-521.

FIG. 4 depicts peptides used to map immunogenic epitopes of HCV NS5A and NS3.

FIG. 5 depicts peptide mapping of immunogenic epitopes of HCV NS5A and NS3.

FIG. 6 depicts NS3- and NS5b-specific CD4+ and CD8+ T cell immunity in mice.

FIG. 7 depicts Kyte-Doolittle hydropathy plots for ActA-N100 fusions with HCV core, NS3, and NS5b antigens based on the genotype 1 consensus sequence.

FIG. 8 depicts antigen expression by Listeria of various ActA-N100 HCV antigen fusions. Panel A shows core sequences 1-190, 1-180, and 1-177 in lanes 3, 4, and 5. Panel B shows NS3 sequences 1-631, 1-484, 22-631, 22-484, 22-280, 172-484, 172-631, and 416-631 in lanes 3-10. Panel C shows NS5 sequences 1-574, 1-342, 320-591, and 320-574 in lanes 3-7. Lanes 1 and 2 in each panel are negative and positive controls showing no antigen insert and mesothelin expression by Listeria monocytogenes CRS-207.

### DETAILED DESCRIPTION

The present invention relates to compositions and methods for delivery of active immunotherapy using a bacterium encoding and expressing one or more hepatitis C virus (HCV) antigens.

It is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### 1. Definitions

Abbreviations used to indicate a mutation in a gene, or a mutation in a bacterium comprising the gene, are as follows. By way of example, the abbreviation “L. monocytogenes ΔActA” means that part, or all, of the ActA gene was deleted. The delta symbol (Δ) means deletion. An abbreviation including a superscripted minus sign (Listeria ActA⁻) means that the ActA gene was mutated, e.g., by way of a deletion, point mutation, or frameshift mutation, but not limited to these types of mutations.

“Administration” as it applies to a human, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” also encompasses in vitro and ex vivo treatments, e.g., of a cell, by a reagent, diagnostic, binding composition, or by another cell.

An “agonist,” as it relates to a ligand and receptor, comprises a molecule, combination of molecules, a complex, or a combination of reagents, that stimulates the receptor. For example, an agonist of granulocyte-macrophage colony stimulating factor (GM-CSF) can encompass GM-CSF, a mutein or derivative of GM-CSF, a peptide mimetic of GM-CSF, a small molecule that mimics the biological function of GM-CSF, or an antibody that stimulates GM-CSF receptor.

An “antagonist,” as it relates to a ligand and receptor, comprises a molecule, combination of molecules, or a complex, that inhibits, counteracts, downregulates, and/or desensitizes the receptor. “Antagonist” encompasses any reagent that inhibits a constitutive activity of the receptor. A constitutive activity is one that is manifest in the absence of a ligand/receptor interaction. “Antagonist” also encompasses any reagent that inhibits or prevents a stimulated (or regulated) activity of a receptor. By way of example, an antagonist of GM-CSF receptor includes, without implying any limitation, an antibody that binds to the ligand (GM-CSF) and prevents it from binding to the receptor, or an antibody that binds to the receptor and prevents the ligand from binding to the receptor, or where the antibody locks the receptor in an inactive conformation.

As used herein, an “analog” with reference to a peptide, polypeptide or protein refers to another peptide, polypeptide or protein that possesses a similar or identical function as the original peptide, polypeptide or protein, but does not necessarily comprise a similar or identical amino acid sequence or structure of the original peptide, polypeptide or protein. An analog preferably satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the original amino acid sequence (b) a proteinaceous agent encoded by a nucleotide sequence that hybridizes under stringent conditions to a nucleotide sequence encoding the original amino acid sequence; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding the original amino acid sequence.

“Antigen presenting cells” (APCs) are cells of the immune system used for presenting antigen to T cells. APCs include dendritic cells, monocytes, macrophages, marginal zone Kupffer cells, microglia, Langerhans cells, T cells, and B cells. Dendritic cells occur in at least two lineages. The first lineage encompasses pre-DC1, myeloid DC1, and mature DC1. The second lineage encompasses CD34⁺⁺CD45RA⁻ early progenitor multipotent cells, CD34⁺⁺CD45RA⁺ cells, CD34⁺⁺CD45RA⁺⁺CD4⁺ IL-3Rα⁺⁺ pro-DC2 cells, CD4⁺CD11c⁻ plasmacytoid pre-DC2 cells, lymphoid human DC2 plasmacytoid-derived DC2s, and mature DC2s.

“Attenuation” and “attenuated” encompasses a bacterium, virus, parasite, infectious organism, prion, tumor cell, gene in the infectious organism, and the like, that is modified to reduce toxicity to a host. The host can be a human or animal host, or an organ, tissue, or cell. The bacterium, to give a non-limiting example, can be attenuated to reduce binding to a host cell, to reduce spread from one host cell to another host cell, to reduce extracellular growth, or to reduce intracellular growth in a host cell. Attenuation can be assessed by measuring, e.g., an indicum or indicia of toxicity, the LD₅₀, the rate of clearance from an organ, or the competitive index (see, e.g., Auerbuch, et al. (2001) Infect. Immunity 69:5953-5957). Generally, an attenuation results an increase in the LD₅₀ and/or an increase in the rate of clearance by at least 25%; more generally by at least 50%; most generally by at least 100% (2-fold); normally by at least 5-fold; more normally by at least 10-fold; most normally by at least 50-fold; often by at least 100-fold; more often by at least 500-fold; and most often by at least 1000-fold; usually by at least 5000-fold; more usually by at least 10,000-fold; and most usually by at least 50,000-fold; and most often by at least 100,000-fold.

“Attenuated gene” encompasses a gene that mediates toxicity, pathology, or virulence, to a host, growth within the host, or survival within the host, where the gene is mutated in a way that mitigates, reduces, or eliminates the toxicity, pathology, or virulence. The reduction or elimination can be assessed by comparing the virulence or toxicity mediated by the mutated gene with that mediated by the non-mutated (or parent) gene. “Mutated gene” encompasses deletions, point mutations, and frameshift mutations in regulatory regions of the gene, coding regions of the gene, non-coding regions of the gene, or any combination thereof.

“Conservatively modified variants” applies to both amino acid and nucleic acid sequences. With respect to particular nucleic acid sequences, a conservatively modified variant refers to nucleic acids encoding identical amino acid sequences, or amino acid sequences that have one or more conservative substitutions. An example of a conservative substitution is the exchange of an amino acid in one of the following groups for another amino acid of the same group (U.S. Pat. No. 5,767,063 issued to Lee, et al.; Kyte and Doolittle (1982) J. Mol. Biol. 157:105-132).

### (1) Hydrophobic: Norleucine, Ile, Val, Leu, Phe, Cys, Met;

(2) Neutral hydrophilic: Cys, Ser, Thr;

### (3) Acidic: Asp, Glu;

### (4) Basic: Asn, Gln, His, Lys, Arg;

(5) Residues that influence chain orientation: Gly, Pro;

### (6) Aromatic: Tip, Tyr, Phe; and

(7) Small amino acids: Gly, Ala, Ser.

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition.

An “extracellular fluid” encompasses, e.g., serum, plasma, blood, interstitial fluid, cerebrospinal fluid, secreted fluids, lymph, bile, sweat, fecal matter, and urine. An “extracelluar fluid” can comprise a colloid or a suspension, e.g., whole blood or coagulated blood.

The term “fragments” in the context of polypeptides include a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a larger polypeptide.

“Gene” refers to a nucleic acid sequence encoding an oligopeptide or polypeptide. The oligopeptide or polypeptide can be biologically active, antigenically active, biologically inactive, or antigenically inactive, and the like. The term gene encompasses, e.g., the sum of the open reading frames (ORFs) encoding a specific oligopeptide or polypeptide; the sum of the ORFs plus the nucleic acids encoding introns; the sum of the ORFs and the operably linked promoter(s); the sum of the ORFS and the operably linked promoter(s) and any introns; the sum of the ORFS and the operably linked promoter(s), intron(s), and promoter(s), and other regulatory elements, such as enhancer(s). In certain embodiments, “gene” encompasses any sequences required in cis for regulating expression of the gene. The term gene can also refer to a nucleic acid that encodes a peptide encompassing an antigen or an antigenically active fragment of a peptide, oligopeptide, polypeptide, or protein. The term gene does not necessarily imply that the encoded peptide or protein has any biological activity, or even that the peptide or protein is antigenically active. A nucleic acid sequence encoding a non-expressable sequence is generally considered a pseudogene. The term gene also encompasses nucleic acid sequences encoding a ribonucleic acid such as rRNA, tRNA, or a ribozyme.

“Growth” of a bacterium such as Listeria encompasses, without limitation, functions of bacterial physiology and genes relating to colonization, replication, increase in protein content, and/or increase in lipid content. Unless specified otherwise explicitly or by context, growth of a Listeria encompasses growth of the bacterium outside a host cell, and also growth inside a host cell. Growth related genes include, without implying any limitation, those that mediate energy production (e.g., glycolysis, Krebs cycle, cytochromes), anabolism and/or catabolism of amino acids, sugars, lipids, minerals, purines, and pyrimidines, nutrient transport, transcription, translation, and/or replication. In some embodiments, “growth” of a Listeria bacterium refers to intracellular growth of the Listeria bacterium, that is, growth inside a host cell such as a mammalian cell. While intracellular growth of a Listeria bacterium can be measured by light microscopy or colony forming unit (CFU) assays, growth is not to be limited by any technique of measurement. Biochemical parameters such as the quantity of a listerial antigen, listerial nucleic acid sequence, or lipid specific to the Listeria bacterium, can be used to assess growth. In some embodiments, a gene that mediates growth is one that specifically mediates intracellular growth. In some embodiments, a gene that specifically mediates intracellular growth encompasses, but is not limited to, a gene where inactivation of the gene reduces the rate of intracellular growth but does not detectably, substantially, or appreciably, reduce the rate of extracellular growth (e.g., growth in broth), or a gene where inactivation of the gene reduces the rate of intracellular growth to a greater extent than it reduces the rate of extracellular growth. To provide a non-limiting example, in some embodiments, a gene where inactivation reduces the rate of intracellular growth to a greater extent than extracellular growth encompasses the situation where inactivation reduces intracellular growth to less than 50% the normal or maximal value, but reduces extracellular growth to only 1-5%, 5-10%, or 10-15% the maximal value. The invention, in certain aspects, encompasses a Listeria attenuated in intracellular growth but not attenuated in extracellular growth, a Listeria not attenuated in intracellular growth and not attenuated in extracellular growth, as well as a Listeria not attenuated in intracellular growth but attenuated in extracellular growth.

A “hydropathy analysis” refers to the analysis of a polypeptide sequence by the method of Kyte and Doolittle: “A Simple Method for Displaying the Hydropathic Character of a Protein”. J. Mol. Biol. 157 (1982)105-132. In this method, each amino acid is given a hydrophobicity score between 4.6 and −4.6. A score of 4.6 is the most hydrophobic and a score of −4.6 is the most hydrophilic. Then a window size is set. A window size is the number of amino acids whose hydrophobicity scores will be averaged and assigned to the first amino acid in the window. The calculation starts with the first window of amino acids and calculates the average of all the hydrophobicity scores in that window. Then the window moves down one amino acid and calculates the average of all the hydrophobicity scores in the second window. This pattern continues to the end of the protein, computing the average score for each window and assigning it to the first amino acid in the window. The averages are then plotted on a graph. The y axis represents the hydrophobicity scores and the x axis represents the window number. The following hydrophobicity scores are used for the 20 common amino acids.

| | | | | | |
|---|---|---|---|---|---|
| Arg: | −4.5 | Ser: | −0.8 | Lys: | −3.9 |
| Thr: | −0.7 | Asn: | −3.5 | Gly: | −0.4 |
| Asp: | −3.5 | Ala: | 1.8 | Gln: | −3.5 |
| Met: | 1.9 | Glu: | −3.5 | Cys: | 2.5 |
| His: | −3.2 | Phe: | 2.8 | Pro: | −1.6 |
| Leu: | 3.8 | Tyr: | −1.3 | Val: | 4.2 |
| Trp: | −0.9 | Ile: | 4.5 | | |

A composition that is “labeled” is detectable, either directly or indirectly, by spectroscopic, photochemical, biochemical, immunochemical, isotopic, or chemical methods. For example, useful labels include ³²P, ³³P, ³⁵S, ₁₄C, ³H, ¹²⁵I, stable isotopes, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

“Ligand” refers to a small molecule, peptide, polypeptide, or membrane associated or membrane-bound molecule, that is an agonist or antagonist of a receptor. “Ligand” also encompasses a binding agent that is not an agonist or antagonist, and has no agonist or antagonist properties. By convention, where a ligand is membrane-bound on a first cell, the receptor usually occurs on a second cell. The second cell may have the same identity (the same name), or it may have a different identity (a different name), as the first cell. A ligand or receptor may be entirely intracellular, that is, it may reside in the cytosol, nucleus, or in some other intracellular compartment. The ligand or receptor may change its location, e.g., from an intracellular compartment to the outer face of the plasma membrane. The complex of a ligand and receptor is termed a “ligand receptor complex.” Where a ligand and receptor are involved in a signaling pathway, the ligand occurs at an upstream position and the receptor occurs at a downstream position of the signaling pathway.

“Nucleic acid” refers to deoxyribonucleotides or ribonucleotides and polymers thereof in either single stranded, double-stranded form, or multi-stranded form. Non-limiting examples of a nucleic acid are a, e.g., cDNA, mRNA, oligonucleotide, and polynucleotide. A particular nucleic acid sequence can also implicitly encompasses “allelic variants” and “splice variants.”

“Operably linked” in the context of a promoter and a nucleic acid encoding a mRNA means that the promoter can be used to initiate transcription of that nucleic acid.

The terms “percent sequence identity” and “% sequence identity” refer to the percentage of sequence similarity found by a comparison or alignment of two or more amino acid or nucleic acid sequences. Percent identity can be determined by a direct comparison of the sequence information between two molecules by aligning the sequences, counting the exact number of matches between the two aligned sequences, dividing by the length of the shorter sequence, and multiplying the result by 100. An algorithm for calculating percent identity is the Smith-Waterman homology search algorithm (see, e.g., Kann and Goldstein (2002) Proteins 48:367-376; Arslan, et al. (2001) Bioinformatics 17:327-337).

By “purified” and “isolated” is meant, when referring to a polypeptide, that the polypeptide is present in the substantial absence of the other biological macromolecules with which it is associated in nature. The term “purified” as used herein means that an identified polypeptide often accounts for at least 50%, more often accounts for at least 60%, typically accounts for at least 70%, more typically accounts for at least 75%, most typically accounts for at least 80%, usually accounts for at least 85%, more usually accounts for at least 90%, most usually accounts for at least 95%, and conventionally accounts for at least 98% by weight, or greater, of the polypeptides present. The weights of water, buffers, salts, detergents, reductants, protease inhibitors, stabilizers (including an added protein such as albumin), and excipients, and molecules having a molecular weight of less than 1000, are generally not used in the determination of polypeptide purity. See, e.g., discussion of purity in U.S. Pat. No. 6,090,611 issued to Covacci, et al.

“Peptide” refers to a short sequence of amino acids, where the amino acids are connected to each other by peptide bonds. A peptide may occur free or bound to another moiety, such as a macromolecule, lipid, oligo- or polysaccharide, and/or a polypeptide. Where a peptide is incorporated into a polypeptide chain, the term “peptide” may still be used to refer specifically to the short sequence of amino acids. A “peptide” may be connected to another moiety by way of a peptide bond or some other type of linkage. A peptide is at least two amino acids in length and generally less than about 25 amino acids in length, where the maximal length is a function of custom or context. The terms “peptide” and “oligopeptide” may be used interchangeably.

“Protein” generally refers to the sequence of amino acids comprising a polypeptide chain. Protein may also refer to a three dimensional structure of the polypeptide. “Denatured protein” refers to a partially denatured polypeptide, having some residual three dimensional structure or, alternatively, to an essentially random three dimensional structure, i.e., totally denatured. The invention encompasses reagents of, and methods using, polypeptide variants, e.g., involving glycosylation, phosphorylation, sulfation, disulfide bond formation, deamidation, isomerization, cleavage points in signal or leader sequence processing, covalent and non-covalently bound cofactors, oxidized variants, and the like. The formation of disulfide linked proteins is described (see, e.g., Woycechowsky and Raines (2000) Curr. Opin. Chem. Biol. 4:533-539; Creighton, et al. (1995) Trends Biotechnol. 13:18-23).

“Recombinant” when used with reference, e.g., to a nucleic acid, cell, animal, virus, plasmid, vector, or the like, indicates modification by the introduction of an exogenous, non-native nucleic acid, alteration of a native nucleic acid, or by derivation in whole or in part from a recombinant nucleic acid, cell, virus, plasmid, or vector. Recombinant protein refers to a protein derived, e.g., from a recombinant nucleic acid, virus, plasmid, vector, or the like. “Recombinant bacterium” encompasses a bacterium where the genome is engineered by recombinant methods, e.g., by way of a mutation, deletion, insertion, and/or a rearrangement. “Recombinant bacterium” also encompasses a bacterium modified to include a recombinant extra-genomic nucleic acid, e.g., a plasmid or a second chromosome, or a bacterium where an existing extra-genomic nucleic acid is altered.

“Sample” refers to a sample from a human, animal, placebo, or research sample, e.g., a cell, tissue, organ, fluid, gas, aerosol, slurry, colloid, or coagulated material. The “sample” may be tested in vivo, e.g., without removal from the human or animal, or it may be tested in vitro. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

A “selectable marker” encompasses a nucleic acid that allows one to select for or against a cell that contains the selectable marker. Examples of selectable markers include, without limitation, e.g.: (1) A nucleic acid encoding a product providing resistance to an otherwise toxic compound (e.g., an antibiotic), or encoding susceptibility to an otherwise harmless compound (e.g., sucrose); (2) A nucleic acid encoding a product that is otherwise lacking in the recipient cell (e.g., tRNA genes, auxotrophic markers); (3) A nucleic acid encoding a product that suppresses an activity of a gene product; (4) A nucleic acid that encodes a product that can be readily identified (e.g., phenotypic markers such as beta-galactosidase, green fluorescent protein (GFP), cell surface proteins, an epitope tag, a FLAG tag); (5) A nucleic acid that can be identified by hybridization techniques, for example, PCR or molecular beacons.

“Specifically” or “selectively” binds, when referring to a ligand/receptor, nucleic acid/complementary nucleic acid, antibody/antigen, or other binding pair (e.g., a cytokine to a cytokine receptor) indicates a binding reaction which is determinative of the presence of the protein in a heterogeneous population of proteins and other biologics. Thus, under designated conditions, a specified ligand binds to a particular receptor and does not bind in a significant amount to other proteins present in the sample. Specific binding can also mean, e.g., that the binding compound, nucleic acid ligand, antibody, or binding composition derived from the antigen-binding site of an antibody, of the contemplated method binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound.

In a typical embodiment an antibody will have an affinity that is greater than about 10⁹ liters/mol, as determined, e.g., by Scatchard analysis (Munsen, et al. (1980) Analyt. Biochem. 107:220-239). It is recognized by the skilled artisan that some binding compounds can specifically bind to more than one target, e.g., an antibody specifically binds to its antigen, to lectins by way of the antibody's oligosaccharide, and/or to an Fc receptor by way of the antibody's Fc region.

“Spread” of a bacterium encompasses “cell to cell spread,” that is, transmission of the bacterium from a first host cell to a second host cell, as mediated, for example, by a vesicle. Functions relating to spread include, but are not limited to, e.g., formation of an actin tail, formation of a pseudopod-like extension, and formation of a double-membraned vacuole.

The term “subject” as used herein refers to a human or non-human organism. Thus, the methods and compositions described herein are applicable to both human and veterinary disease. In certain embodiments, subjects are “patients,” i.e., living humans that are receiving medical care for a disease or condition. This includes persons with no defined illness who are being investigated for signs of pathology. Preferred are subjects who have an existing HCV infection, most preferably a chronic infection.

The “target site” of a recombinase is the nucleic acid sequence or region that is recognized, bound, and/or acted upon by the recombinase (see, e.g., U.S. Pat. No. 6,379,943 issued to Graham, et al.; Smith and Thorpe (2002) Mol. Microbiol. 44:299-307; Groth and Calos (2004) J. Mol. Biol. 335:667-678; Nunes-Duby, et al. (1998) Nucleic Acids Res. 26:391-406).

“Therapeutically effective amount” is defined as an amount of a reagent or pharmaceutical composition that is sufficient to show a patient benefit, i.e., to cause a decrease, prevention, or amelioration of the symptoms of the condition being treated. When the agent or pharmaceutical composition comprises a diagnostic agent, a “diagnostically effective amount” is defined as an amount that is sufficient to produce a signal, image, or other diagnostic parameter. Effective amounts of the pharmaceutical formulation will vary according to factors such as the degree of susceptibility of the individual, the age, gender, and weight of the individual, and idiosyncratic responses of the individual (see, e.g., U.S. Pat. No. 5,888,530 issued to Netti, et al.).

“Treatment” or “treating” (with respect to a condition or a disease) is an approach for obtaining beneficial or desired results including and preferably clinical results. For purposes of this invention, beneficial or desired results with respect to a disease include, but are not limited to, one or more of the following: improving a condition associated with a disease, curing a disease, lessening severity of a disease, delaying progression of a disease, alleviating one or more symptoms associated with a disease, increasing the quality of life of one suffering from a disease, and/or prolonging survival. Likewise, for purposes of this invention, beneficial or desired results with respect to a condition include, but are not limited to, one or more of the following: improving a condition, curing a condition, lessening severity of a condition, delaying progression of a condition, alleviating one or more symptoms associated with a condition, increasing the quality of life of one suffering from a condition, and/or prolonging survival. For instance, in some embodiments where the compositions described herein are used for treatment of cancer, the beneficial or desired results include, but are not limited to, one or more of the following: reducing the proliferation of (or destroying) neoplastic or cancerous cells, reducing metastasis of neoplastic cells found in cancers, shrinking the size of a tumor, decreasing symptoms resulting from the cancer, increasing the quality of life of those suffering from the cancer, decreasing the dose of other medications required to treat the disease, delaying the progression of the cancer, and/or prolonging survival of patients having cancer. Depending on the context, “treatment” of a subject can imply that the subject is in need of treatment, e.g., in the situation where the subject comprises a disorder expected to be ameliorated by administration of a reagent.

“Vaccine” encompasses preventative vaccines. Vaccine also encompasses therapeutic vaccines, e.g., a vaccine administered to a mammal that comprises a condition or disorder associated with the antigen or epitope provided by the vaccine.

### 2. Hepatitis C Antigens

HCV has a positive-stranded RNA genome containing a large open reading frame which encodes a precursor polyprotein of about 3,000 amino acids. This polyprotein is cleaved by host and viral proteases into 10 viral proteins, referred to as core, E1, E2, p7, NS2, NS3, NS4a, NS4b, NS5a, and NS5b antigens. While the following examples address the use of core, NS5b and NS3, any one or more of these HCV antigen sequences may find use in the vaccine compositions and methods described herein.

As used herein, the term “HCV antigen” refers to a polypeptide encoding an amino acid sequence which comprises (i) one or more full length HCV proteins selected from the group consisting of core, E1, E2, p7, NS2, NS3, NS4a, NS4b, NS5a, and/or NS5b; and/or (ii) one or more polypeptide sequences derived from one or more full length HCV proteins independently selected from the group consisting of core, E1, E2, p7, NS2, NS3, NS4a, NS4b, NS5a, and/or NS5b. The HCV antigens described herein may be used individually, but are preferably used in combinations comprising polypeptide sequences from at least two, three, four, five, or more HCV proteins selected from the group consisting of core, E1, E2, p7, NS2, NS3, NS4a, NS4b, NS5a, and/or NS5b. As described hereinafter, preferred HCV antigens comprise NS3 and/or NS5b polypeptide sequences or sequences derived therefrom.

As noted, the HCV antigen(s) used in the present invention may comprise full length versions of core, E1, E2, p7, NS2, NS3, NS4a NS4b, NS5a, and/or NS5b antigens, or may comprise sequences “derived from” one or more such full length HCV antigens. By “derived from” as used herein is meant a polypeptide having one or more conservative amino acid changes as compared to a specified HCV antigen or antigens, a polypeptide comprising one or more isolated epitopes from a specified HCV antigen or antigens, or a peptide or polypeptide that is immunologically cross reactive with a specified HCV antigen or antigen.

In some embodiments, an antigen that is “derived from” an HCV antigen comprises a partial sequence (“a fragment”) of one or more full length HCV antigens. Thus, an “HCV antigen” can refer to a polypeptide encoding an amino acid sequence comprising (i) one or more full length HCV proteins selected from the group consisting of core E1, E2, p7, NS2, NS3, NS4a, NS4b, NS5a, and/or NS5b; and/or (ii) one or more partial polypeptide sequences of one or more HCV proteins independently selected from the group consisting of core, E1, E2, p7, NS2, NS3, NS4a, NS4b, NS5a, and/or NS5b. In various embodiments, a derived HCV antigen comprises a fragment of at least 8 amino acids, at least 12 amino acids, at least 20 amino acids, at least 50 amino acids, at least 75 amino acids, at least 100 amino acids, or at least 200 amino acids or more, obtained from a full length HCV antigen.

The antigen can comprise a sequence encoding at least one MHC class I epitope and/or at least one MHC class II epitope obtained from an original (full-length) HCV antigen. Publicly available algorithms can be used to select epitopes that bind to MHC class I and/or class II molecules. For example, the predictive algorithm “BIMAS” ranks potential HLA binding epitopes according to the predictive half-time disassociation of peptide/HLA complexes. The “SYFPEITHI” algorithm ranks peptides according to a score that accounts for the presence of primary and secondary HLA-binding anchor residues. Both computerized algorithms score candidate epitopes based on amino acid sequences within a given protein that have similar binding motifs to previously published HLA binding epitopes. Other algorithms can also be used to identify candidates for further biological testing.

The derivative of an antigen may also comprise an amino acid sequence which has at least about 80% sequence identity, at least about 85% sequence identity, at least about 90% sequence identity, at least about 95% sequence identity, or at least about 98% sequence identity to the portion of the HCV antigen from which it is derived. Preferably the HCV antigen expressed by the vaccine construct differs from the wild type equivalent in that the antigen comprises one or more mutations engineered into motifs critical for one or more functions of each protein. For example, in the case of NS5b from the genotype 1 consensus sequence, a GDD to GNH (beginning at amino acid 317 of NS5b) inactivating double mutation completely inactivates RNA polymerase activity. Likewise, in the case of NS3 from the genotype 1 consensus sequence, a mutation of motif II (DECH) to AASH beginning at amino acid 290 abolishes helicase activity.

There are at least 6 known genotypes and more than 50 subtypes of HCV. While the following examples address the use of genotype 1 antigens, the methods and compositions described herein are applicable to all HCV genotypes and subtypes. Thus, one or more antigen sequences for use in the present invention may be obtained from any specific HCV genotype/subtype.

Neither a prophylactic nor a therapeutic HCV vaccine is currently available, and a significant challenge to the development of a vaccine is the underlying diversity of the virus. HCV is highly diverse both between and within persons as it exists in each infected person as a quasispecies, or “swarm” of closely related but distinct genetic sequences. Thus, one strategy in the development of an effective HCV vaccine is to obtain one or more antigens, not from an individual subtype, but from a consensus sequence for a particular genotype which is based upon the most commonly found amino acids at each position for a given antigen. See, e.g., WO06/086188, which is hereby incorporated by reference in its entirety, including all tables, figures, and claims. One theoretical advantage of the consensus sequence is that it minimizes the genetic differences between vaccine strains and contemporary isolates, effectively reducing the extent of diversity by half, and thus it may have enhanced potential for eliciting cross-reactive responses. A consensus sequence vaccine would also be far more efficient to produce because the consensus is unlikely to vary among geographic regions.

Thus, in preferred embodiments, the HCV antigen used is based on an HCV consensus sequence. For example, an HCV antigen may be a polypeptide encoding an amino acid sequence comprising (i) a consensus sequence of one or more full length HCV proteins selected from the group consisting of core, E1, E2, p7, NS2, NS3, NS4a, NS4b, NS5a, and/or NS5b; and/or (ii) one or more polypeptide sequences derived from a consensus sequence of one or more full length HCV proteins independently selected from the group consisting of core, E1, E2, p7, NS2, NS3, NS4a, NS4b, NS5a, and/or NS5b. The following table provides Swiss-Prot entry data for a variety of HCV isolates:

| | |
|---|---|
| POLG_HCV1 (P26664) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 1a (isolate 1) (HCV) |
| POLG_HCV6A (Q5I2N3) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 6a (isolate 6a33) (HCV) |
| POLG_HCVBB (Q68749) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 2c (isolate BEBE1) (HCV) |
| POLG_HCVBK (P26663) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 1b (isolate BK) (HCV) |
| POLG_HCVCO (Q9WMX2) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 1b (isolate Con1) (HCV) |
| POLG_HCVED (O39929) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 4a (isolate ED43) (HCV) |
| POLG_HCVEU (O39927) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 6a (isolate EUHK2) (HCV) |
| POLG_HCVEV (O39928) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 5a (isolate EUH1480) (HCV) |
| POLG_HCVH (P27958) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 1a (isolate H) (HCV) |
| POLG_HCVH9 (Q81754) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 1c (isolate HC-G9) (HCV) |
| POLG_HCVIN (Q913D4) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 1c (isolate India) (HCV) |
| POLG_HCVJ1 (Q03463) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 1b (isolate HC-J1) (HCV) |
| POLG_HCVJ4 (O92972) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 1b (strain HC-J4) (HCV) |
| POLG_HCVJ6 (P26660) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 2a (isolate HC-J6) (HCV) |
| POLG_HCVJ8 (P26661) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 2b (isolate HC-J8) (HCV) |
| POLG_HCVJA (P26662) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 1b (isolate Japanese) (HCV) |
| POLG_HCVJF (Q99IB8) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 2a (isolate JFH-1) (HCV) |
| POLG_HCVJK (Q68801) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 3k (isolate JK049) (HCV) |
| POLG_HCVJL (Q68798) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 6g (isolate JK046) (HCV) |
| POLG_HCVJP (Q9DHD6) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 2b (isolate JPUT971017) (HCV) |
| POLG_HCVJT (Q00269) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 1b (isolate HC-JT) (HCV) |
| POLG_HCVK3 (Q81495) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 3a (isolate k3a) (HCV) |
| POLG_HCVNZ (Q81258) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 3a (isolate NZL1) (HCV) |
| POLG_HCVR6 (Q913V3) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 1b (isolate HCR6) (HCV) |
| POLG_HCVSA (O91936) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 5a (isolate SA13) (HCV) |
| POLG_HCVT5 (O92529) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 6b (isolate Th580) (HCV) |
| POLG_HCVTR (Q81487) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 3b (isolate Tr-Kj) (HCV) |
| POLG_HCVTW (P29846) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 1b (isolate Taiwan) (HCV) |
| POLG_HCVVA (Q9QAX1) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 2k (isolate VAT96) (HCV) |
| POLG_HCVVN (O92530) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 6d (isolate VN235) (HCV) |
| POLG_HCVVO (O92531) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 6k (isolate VN405) (HCV) |
| POLG_HCVVP (O92532) | Genome polyprotein [Contains: Core protein p21 (Capsid |
| | protein C) (p21); Core protein p19; Envelope glycoprotein |
| | E1 (gp32) (gp35); Envelope glycoprotein E2 (NS1) (gp68) |
| | (gp70); p7; Protease NS2-3 (EC 3.4.22.—) (p23); Serine |
| | protease/NTPase/helicase NS3 (EC 3.4.21.98) (EC 3.6.1.15) |
| | (EC 3.6.1.—) (Hepacivirin) (NS3P) (p70); Non-structural |
| | protein 4A (NS4A) (p8); Non-structural protein 4B (NS4B) |
| | (p27); Non-structural protein 5A (NS5A) (p56); RNA- |
| | directed RNA polymerase (EC 2.7.7.48) (NS5B) (p68)] - |
| | Hepatitis C virus genotype 6h (isolate VN004) (HCV) |

The following consensus sequences, while preferred, are exemplary in nature and should not be considered limiting:

| Core; consensus genotype 1a | |
|---|---|
| (SEQ ID NO: 7) | |
| MSTNPKPQRK TKRNTNRRPQ DVKFPGGGQI VGGVYLLPRR GPRLGVRATR | 50 |
| KTSERSQPRG RRQPIPKARR PEGRTWAQPG YPWPLYGNEG CGWAGWLLSP | 100 |
| RGSRPSWGPT DPRRRSRNLG KVIDTLTCGF ADLMGYIPLV GAPLGGAARA | 150 |
| LAHGVRVLED GVNYATGNLP GCSFSIFLLA LLSCLTVPAS A | 191 |
| Core; consensus genotype 1b | |
| (SEQ ID NO: 8) | |
| MSTNPKPQRK TKRNTNRRPQ DVKFPGGGQI VGGVYLLPRR GPRLGVRATR | 50 |
| KTSERSQPRG RRQPIPKARR PEGRAWAQPG YPWPLYGNEG MGWAGWLLSP | 100 |
| RGSRPSWGPT DPRRRSRNLG KVIDTLTCGF ADLMGYIPLV GAPLGGAARA | 150 |
| LAHGVRVLED GVNYATGNLP GCSFSIFLLA LLSCLTIPAS A | 191 |
| E1; consensus genotype 1a | |
| (SEQ ID NO: 9) | |
| YQVRNSSGLY HVTNDCPNSS VVYEAADAIL HTPGCVPCVR EGNASRCWVA | 50 |
| VTPTVATRDG KLPTTQLRRH IDLLVGSATL CSALYVGDLC GSVFLVGQLF | 100 |
| TFSPRHHWTT QDCNCSIYPG HITGHRMAWN MMMNWSPTAA LVVAQLLRIP | 150 |
| QAIMDMIAGA HWGVLAGIKY FSMVGNWAKV LVVLLLFAGV DA | 192 |
| E2; consensus genotype 1a | |
| (SEQ ID NO: 10) | |
| ETHVTGGNAG RTTAGLVGLL TPGAKQNIQL INTNGSWHIN STALNCNESL | 50 |
| NTGWLAGLFY QHKFNSSGCP ERLASCRRLT DFAQGWGPIS YANGSGLDER | 100 |
| PYCWHYPPRP CGIVPAKSVC GPVYCFTPSP VVVGTTDRSG APTYSWGAND | 150 |
| TDVFVLNNTR PPLGNWFGCT WMNSTGFTKV CGAPPCVIGG VGNNTLLCPT | 200 |
| DCFRKYPEAT YSRCGSGPRI TPRCMVDYPY RLWHYPCTIN YTIFKVRMYV | 250 |
| GGVEHRLEAA CNWTRGERCD LEDRDRSELS PLLLSTTQWQ VLPCSFTTLP | 300 |
| ALSTGLIHLH QNIVDVQYLY GVGSSIASWA IKWEYVVLLF LLLADARVCS | 350 |
| CLWMMLLISQ AEA | 363 |
| p7; consensus genotype 1a | |
| (SEQ ID NO: 11) | |
| ALENLVILNA ASLAGTHGLV SFLVFFCFAW YLKGRWVPGA VYALYGMWPL | 50 |
| LLLLLALPQR AYA | 63 |
| NS2; consensus genotype 1a | |
| (SEQ ID NO: 12) | |
| LDTEVAASCG GVVLVGLMAL TLSPYYKRYI SWCMWWLQYF LTRVEAQLHV | 50 |
| WVPPLNVRGG RDAVILLTCV VHPALVFDIT KLLLAIFGPL WILQASLLKV | 100 |
| PYFVRVQGLL RICALARKIA GGHYVQMAII KLGALTGTCV YNHLAPLRDW | 150 |
| AHNGLRDLAV AVEPVVFSRM ETKLITWGAD TAACGDIING LPVSARRGQE | 200 |
| ILLGPADGMV SKGWRLL | 217 |
| NS3; consensus genotype 1a | |
| (SEQ ID NO: 13) | |
| APITAYAQQT RGLLGCIITS LTGRDKNQVE GEVQIVSTAA QTFLATCING | 50 |
| VCWTVYHGAG TRTIASSKGP VIQMYTNVDQ DLVGWPAPQG ARSLTPCTCG | 100 |
| SSDLYLVTRH ADVIPVRRRG DSRGSLLSPR PISYLKGSSG GPLLCPAGHA | 150 |
| VGIFRAAVCT RGVAKAVDFI PVENLETTMR SPVFTDNSSP PAVPQSFQVA | 200 |
| HLHAPTGSGK STKVPAAYAA QGYKVLVLNP SVAATLGFGA YMSKAHGIDP | 250 |
| NIRTGVRTIT TGSPITYSTY GKFLADGGCS GGAYDIIICD ECHSTDATSI | 300 |
| LGIGTVLDQA ETAGARLVVL ATATPPGSVT VPHPNIEEVA LSTTGEIPFY | 350 |
| GKAIPLEVIK GGRHLIFCHS KKKCDELAAK LVALGINAVA YYRGLDVSVI | 400 |
| PTSGDVVVVA TDALMTGYTG DFDSVIDCNT CVTQTVDFSL DPTFTIETTT | 450 |
| LPQDAVSRTQ RRGRTGRGKP GIYRFVAPGE RPSGMFDSSV LCECYDAGCA | 500 |
| WYELTPAETT VRLRAYMNTP GLPVCQDHLE FWEGVFTGLT HIDAHFLSQT | 550 |
| KQSGENFPYL VAYQATVCAR AQAPPPSWDQ MWKCLIRLKP TLHGPTPLLY | 600 |
| RLGAVQNEVT LTHPVTKYIM TCMSADLEVV T | 631 |
| NS3; consensus genotype 1b | |

| (SEQ ID NO: 14) | |
|---|---|
| APITAYSQQT RGLLGCIITS LTGRDKNQVE GEVQVVSTAT QSFLATCVNG | 50 |
| VCWTVYHGAG SKTLAGPKGP ITQMYTNVDQ DLVGWQAPPG ARSLTPCTCG | 100 |
| SSDLYLVTRH ADVIPVRRRG DSRGSLLSPR PVSYLKGSSG GPLLCPSGHA | 150 |
| VGIFRAAVCT RGVAKAVDFV PVESMETTMR SPVFTDNSSP PAVPQTFQVA | 200 |
| HLHAPTGSGK STKVPAAYAA QGYKVLVLNP SVAATLGFGA YMSKAHGVDP | 250 |
| NIRTGVRTIT TGAPITYSTY GKFLADGGCS GGAYDIIICD ECHSTDSTTI | 300 |
| LGIGTVLDQA ETAGARLVVL ATATPPGSVT VPHPNIEEVA LSNTGEIPFY | 350 |
| GKAIPIETIK GGRHLIFCHS KKKCDELAAK LSGLGLNAVA YYRGLDVSVI | 400 |
| PTSGDVVVVA TDALMTGFTG DFDSVIDCNT CVTQTVDFSL DPTFTIETTT | 450 |
| VPQDAVSRSQ RRGRTGRGRR GIYRFVTPGE RPSGMFDSSV LCECYDAGCA | 500 |
| WYELTPAETS VRLRAYLNTP GLPVCQDHLE FWESVFTGLT HIDAHFLSQT | 550 |
| KQAGDNFPYL VAYQATVCAR AQAPPPSWDQ MWKCLIRLKP TLHGPTPLLY | 600 |
| RLGAVQNEVT LTHPITKYIM ACMSADLEVV T | 631 |
| NS4a; consensus genotype 1a | |
| (SEQ ID NO: 15) | |
| STWVLVGGVL AALAAYCLST GCVVIVGRIV LSGKPAIIPD REVLYQEFDE | 50 |
| MEEC | 54 |
| NS4b; consensus genotype 1a | |
| (SEQ ID NO: 16) | |
| SQHLPYIEQG MMLAEQFKQK ALGLLQTASR HAEVITPAVQ TNWQKLEVFW | 50 |
| AKHMWNFISG IQYLAGLSTL PGNPAIASLM AFTAAVTSPL TTGQTLLFNI | 100 |
| LGGWVAAQLA APGAATAFVG AGLAGAALDS VGLGKVLVDI LAGYGAGVAG | 150 |
| ALVAFKIMSG EVPSTEDLVN LLPAILSPGA LAVGVVFASI LRRRVGPGEG | 200 |
| AVQWMNRLIA FASRGNHVSP THYVPESDAA ARVTAILSSL TVTQLLRRLH | 250 |
| QWISSECTTP C | 261 |
| NS5a; consensus genotype 1a | |
| (SEQ ID NO: 17) | |
| SGSWLRDIWD WICEVLSDFK TWLKAKLMPQ LPGIPFVSCQ RGYRGVWRGD | 50 |
| GIMHTRCHCG AEITGHVKNG TMRIVGPRTC KNMWSGTFFI NAYTTGPCTP | 100 |
| LPAPNYKFAL WRVSAEEYVE IRRVGDFHYV SGMTTDNLKC PCQIPSPEFF | 150 |
| TELDGVRLHR FAPPCKPLLR EEVSFRVGLH EYPVGSQLPC EPEPDVAVLT | 200 |
| SMLTDPSHIT AEAAGRRLAR GSPPSMASSS ASQLSAPSLK ATCTANHDSP | 250 |
| DAELIEANLL WRQEMGGNIT RVESENKVVI LDSFDPLVAE EDEREVSVPA | 300 |
| EILRKSRRFA PALPVWARPD YNPLLVETWK KPDYEPPVVH GCPLPPPRSP | 350 |
| PVPPPRKKRT VVLTESTLPT ALAELATKSF GSSSTSGITG DNTTTSSEPA | 400 |
| PSGCPPDSDV ESYSSMPPLE GEPGDPDLSD GSWSTVSSGA DTEDVVCC | 448 |
| NS5b; consensus genotype 1a | |
| (SEQ ID NO: 18) | |
| SMSYSWTGAL VTPCAAEEQK LPINALSNSL LRHHNLVYST TSRSACQRQK | 50 |
| KVTFDRLQVL DSHYQDVLKE VKAAASKVKA NLLSVEEACS LTPPHSAKSK | 100 |
| FGYGAKDVRC HARKAVNHIN SVWKDLLEDS VTPIDTTIMA KNEVFCVQPE | 150 |
| KGGRKPARLI VFPDLGVRVC EKMALYDVVS KLPLAVMGSS YGFQYSPGQR | 200 |
| VEFLVQAWKS KKTPMGFSYD TRCFDSTVTE SDIRTEEAIY QCCDLDPQAR | 250 |
| VAIKSLTERL YVGGPLTNSR GENCGYRRCR ASGVLTTSCG NTLTCYIKAQ | 300 |
| AACRAAGLRD CTMLVCGDDL VVICESAGVQ EDAASLRAFT EAMTRYSAPP | 350 |
| GDPPQPEYDL ELITSCSSNV SVAHDGAGKR VYYLTRDPTT PLARAAWETA | 400 |
| RHTPVNSWLG NIIMFAPTLW ARMILMTHFF SVLIARDQLE QALDCEIYGA | 450 |
| CYSIEPLDLP PIIQRLHGLS AFSLHSYSPG EINRVAACLR KLGVPPLRAW | 500 |
| RHRARSVRAR LLSRGGRAAI CGKYLFNWAV RTKLKLTPIA AAGQLDLSGW | 550 |
| FTAGYSGGDI YHSVSRARPR WFWFCLLLLA AGVGIYLLPN R | 591 |
| NS5b; consensus genotype 1b | |
| (SEQ ID NO: 19) | |
| SMSYTWTGAL ITPCAAEESK LPINALSNSL LRHHNMVYAT TSRSASQRQK | 50 |
| KVTFDRLQVL DDHYRDVLKE MKAKASTVKA KLLSVEEACK LTPPHSAKSK | 100 |
| FGYGAKDVRN LSSKAVNHIR SVWKDLLEDT ETPIDTTIMA KNEVFCVQPE | 150 |
| KGGRKPARLI VFPDLGVRVC EKMALYDVVS TLPQAVMGSS YGFQYSPGQR | 200 |
| VEFLVNAWKS KKNPMGFAYD TRCFDSTVTE NDIRVEESIY QCCDLAPEAR | 250 |
| QAIRSLTERL YIGGPLTNSK GQNCGYRRCR ASGVLTTSCG NTLTCYLKAS | 300 |
| AACRAAKLQD CTMLVCGDDL VVICESAGTQ EDAASLRVFT EAMTRYSAPP | 350 |
| GDPPQPEYDL ELITSCSSNV SVAHDASGKR VYYLTRDPTT PLARAAWETA | 400 |
| RHTPVNSWLG NIIMYAPTLW ARMILMTHFF SILLAQEQLE KALDCQIYGA | 450 |
| CYSIEPLDLP QIIQRLHGLS AFSLHSYSPG EINRVASCLR KLGVPPLRVW | 500 |
| RHRARSVRAK LLSQGGRAAT CGKYLFNWAV RTKLKLTPIP AASQLDLSGW | 550 |
| FVAGYSGGDI YHSLSRARPR WFMLCLLLLS VGVGIYLLPN R | 591 |
| NS3; genotype 1a DECH -> AASH mutant | |
| (SEQ ID NO: 20) | |
| APITAYAQQT RGLLGCIITS LTGRDKNQVE GEVQIVSTAA QTFLATCING | 50 |
| VCWTVYHGAG TRTIASSKGP VIQMYTNVDQ DLVGWPAPQG ARSLTPCTCG | 100 |
| SSDLYLVTRH ADVIPVRRRG DSRGSLLSPR PISYLKGSSG GPLLCPAGHA | 150 |
| VGIFRAAVCT RGVAKAVDFI PVENLETTMR SPVFTDNSSP PAVPQSFQVA | 200 |
| HLHAPTGSGK STKVPAAYAA QGYKVLVLNP SVAATLGFGA YMSKAHGIDP | 250 |
| NIRTGVRTIT TGSPITYSTY GKFLADGGCS GGAYDIIICA ASHSTDATSI | 300 |
| LGIGTVLDQA ETAGARLVVL ATATPPGSVT VPHPNIEEVA LSTTGEIPFY | 350 |
| GKAIPLEVIK GGRHLIFCHS KKKCDELAAK LVALGINAVA YYRGLDVSVI | 400 |
| PTSGDVVVVA TDALMTGYTG DFDSVIDCNT CVTQTVDFSL DPTFTIETTT | 450 |
| LPQDAVSRTQ RRGRTGRGKP GIYRFVAPGE RPSGMFDSSV LCECYDAGCA | 500 |
| WYELTPAETT VRLRAYMNTP GLPVCQDHLE FWEGVFTGLT HIDAHFLSQT | 550 |
| KQSGENFPYL VAYQATVCAR AQAPPPSWDQ MWKCLIRLKP TLHGPTPLLY | 600 |
| RLGAVQNEVT LTHPVTKYIM TCMSADLEVV T | 631 |
| NS3; genotype 1b DECH -> AASH mutant | |
| (SEQ ID NO: 21) | |
| APITAYSQQT RGLLGCIITS LTGRDKNQVE GEVQVVSTAT QSFLATCVNG | 50 |
| VCWTVYHGAG SKTLAGPKGP ITQMYTNVDQ DLVGWQAPPG ARSLTPCTCG | 100 |
| SSDLYLVTRH ADVIPVRRRG DSRGSLLSPR PVSYLKGSSG GPLLCPSGHA | 150 |
| VGIFRAAVCT RGVAKAVDFV PVESMETTMR SPVFTDNSSP PAVPQSFQVA | 200 |
| HLHAPTGSGK STKVPAAYAA QGYKVLVLNP SVAATLGFGA YMSKAHGVDP | 250 |
| NIRTGVRTIT TGAPITYSTY GKFLADGGCS GGAYDIIICA ASHSTDSTTI | 300 |
| LGIGTVLDQA ETAGARLVVL ATATPPGSVT VPHPNIEEVA LSNTGEIPFY | 350 |
| GKAIPIETIK GGRHLIFCHS KKKCDELAAK LSGLGLNAVA YYRGLDVSVI | 400 |
| PTSGDVVVVA TDALMTGFTG DFDSVIDCNT CVTQTVDFSL DPTFTIETTT | 450 |
| VPQDAVSRSQ RRGRTGRGRR GIYRFVAPGE RPSGMFDSSV LCECYDAGCA | 500 |
| WYELTPAETS VRLRAYLNTP GLPVCQDHLE FWESVFTGLT HIDAHFLSQT | 550 |
| KQAGDNFPYL VAYQATVCAR AQAPPPSWDQ MWKCLIRLKP TLHGPTPLLY | 600 |
| RLGAVQNEVT LTHPITKYIM ACMSADLEVV T | 631 |
| NS5b; genotype 1a GDD -> GNH mutant | |
| (SEQ ID NO: 22) | |
| SMSYSWTGAL VTPCAAEEQK LPINALSNSL LRHHNLVYST TSRSACQRQK | 50 |
| KVTFDRLQVL DSHYQDVLKE VKAAASKVKA NLLSVEEACS LTPPHSAKSK | 100 |
| FGYGAKDVRC HARKAVNHIN SVWKDLLEDS VTPIDTTIMA KNEVFCVQPE | 150 |
| KGGRKPARLI VFPDLGVRVC EKMALYDVVS KLPLAVMGSS YGFQYSPGQR | 200 |
| VEFLVQAWKS KKTPMGFSYD TRCFDSTVTE SDIRTEEAIY QCCDLDPQAR | 250 |
| VAIKSLTERL YVGGPLTNSR GENCGYRRCR ASGVLTTSCG NTLTCYIKAQ | 300 |
| AACRAAGLRD CTMLVCGNLL VVICESAGVQ EDAASLRAFT EAMTRYSAPP | 350 |
| GDPPQPEYDL ELITSCSSNV SVAHDGAGKR VYYLTRDPTT PLARAAWETA | 400 |
| RHTPVNSWLG NIIMFAPTLW ARMILMTHFF SVLIARDQLE QALDCEIYGA | 450 |
| CYSIEPLDLP PIIQRLHGLS AFSLHSYSPG EINRVAACLR KLGVPPLRAW | 500 |
| RHRARSVRAR LLSRGGRAAI CGKYLFNWAV RTKLKLTPIA AAGQLDLSGW | 550 |
| FVAGYSGGDI YHSVSRARPR WFWFCLLLLA AGVGIYLLPN R | 591 |
| NS5b; genotype 1b GDD -> GNH mutant | |
| (SEQ ID NO: 23) | |
| SMSYTWTGAL ITPCAAEESK LPINALSNSL LRHHNMVYAT TSRSASQRQK | 50 |
| KVTFDRLQVL DDHYRDVLKE MKAKASTVKA KLLSVEEACK LTPPHSAKSK | 100 |
| FGYGAKDVRN LSSKAVNHIR SVWKDLLEDT ETPIDTTIMA KNEVFCVQPE | 150 |
| KGGRKPARLI VFPDLGVRVC EKMALYDVVS TLPQAVMGSS YGFQYSPGQR | 200 |
| VEFLVNAWKS KKNPMGFAYD TRCFDSTVTE NDIRVEESIY QCCDLAPEAR | 250 |
| QAIRSLTERL YIGGPLTNSK GQNCGYRRCR ASGVLTTSCG NTLTCYLKAS | 300 |
| AACRAAKLQD CTMLVCGNLL VVICESAGTQ EDAASLRVFT EAMTRYSAPP | 350 |
| GDPPQPEYDL ELITSCSSNV SVAHDASGKR VYYLTRDPTT PLARAAWETA | 400 |
| RHTPVNSWLG NIIMYAPTLW ARMILMTHFF SILLAQEQLE KALDCQIYGA | 450 |
| CYSIEPLDLP QIIQRLHGLS AFSLHSYSPG EINRVASCLR KLGVPPLRVW | 500 |
| RHRARSVRAK LLSQGGRAAT CGKYLFNWAV RTKLKLTPIP AASQLDLSGW | 550 |
| FVAGYSGGDI YHSLSRARPR WFMLCLLLLS VGVGIYLLPN R | 591 |

Selection of one or more antigens or derivatives thereof for use in the vaccine compositions of the present invention may be performed in a variety of ways, including an assessment of the ability of a bacterium of choice to successfully express and secrete the recombinant antigen(s); and/or the ability of the recombinant antigen(s) to initiate an antigen specific CD4+ and/or CD8+ T cell response. As discussed hereinafter, in order to arrive at a final selection of antigen(s) for use with a particular bacterial delivery vehicle, these attributes of the recombinant antigen(s) are preferably combined with the ability of the complete vaccine platform (meaning the selected bacterial expression system for the HCV antigen(s)) to initiate both the innate immune response as well as an antigen-specific T cell response against the recombinantly expressed HCV antige(s).

An initial determination of suitable antigens may be made by by selecting antigen(s) or antigen fragment(s) that are successfully recombinantly expressed by the bacterial host of choice (e.g., Listeria), and that are immunogenic. By “immunogenic” as that term is used herein is meant that the antigen is capable of eliciting an antigen-specific T-cell response (CD4+ and/or CD8+). Preferred HCV antigens or derivatives thereof comprise one or more of the following polypeptide sequences: IPVENLETTMRSPVF (SEQ ID NO: 1); NLETTMRSPVFTDNS (SEQ ID NO: 2); PPAVPQSFQVAHLHA (SEQ ID NO: 3); PQSFQVAHLHAPTGS (SEQ ID NO: 4); FQVAHLHAPTGSGKS (SEQ ID NO: 5). Other preferred HCV antigens or derivatives thereof comprise one or more of the following polypeptide sequences from NS3:

(SEQ ID NO: 42)
LETTMRSPVFTDNSSPPVVP;
(SEQ ID NO: 43)
SPVFTDNSSPPAVPQ;
(SEQ ID NO: 44)
VPQSFQVAHLHAPTG;
(SEQ ID NO: 45)
FQVAHLHAPTGSGKS;
(SEQ ID NO: 46)
KVPAAYAAQGYKVLV;
(SEQ ID NO: 47)
PAAYAAQGYKVLVLNPSVAA;
(SEQ ID NO: 48)
AAKGYKVLVLNPSVA;
(SEQ ID NO: 49)
VLVLNPSVAA;
(SEQ ID NO: 50)
AQGYKVLVLNPSVAA;
(SEQ ID NO: 51)
QGYKVLVLNPSVAA;
(SEQ ID NO: 52)
GYKVLVLNPSVAAT;
(SEQ ID NO: 53)
GYKVLVLNPSVAATLGFGAY;
(SEQ ID NO: 54)
GVRTITTGSPITYSTYGKFL;
(SEQ ID NO: 55)
ITYSTYGKFLADGGCSGGAY;
(SEQ ID NO: 56)
LADAGCSGGAYDIIICDE;
(SEQ ID NO: 57)
GGAYDIIICDECHST;
(SEQ ID NO: 58)
DIIICDECHSTDATS;
(SEQ ID NO: 59)
TDATSILGIGTVLDQAETAG;
(SEQ ID NO: 60)
ATSILGIGTVLDQAE;
(SEQ ID NO: 61)
VIKGGRHLIFCHSKKKCD;
(SEQ ID NO: 62)
GRHLIFCHSKR;
(SEQ ID NO: 63)
KCDELAAKLVALGIN;
(SEQ ID NO: 64)
GINAVAYYRGLDVSVIPTSG;
(SEQ ID NO: 65)
IPTNGDVVVVSTDALMTG;
(SEQ ID NO: 66)
ALMTGYTGDFDSVID;
(SEQ ID NO: 67)
DFDSVIDCNTCVTQTVDF;
(SEQ ID NO: 68)
SVIDCNTCVTQTVDFSLDPT;
(SEQ ID NO: 69)
CNTCVTQTVDFSLDPTFT;
(SEQ ID NO: 70)
NTCVTQTVDFSLDPT;
(SEQ ID NO: 71)
PTFTIETTTLPQDAVSRT;
(SEQ ID NO: 72)
TQTVDFSLDPTFTIE;
(SEQ ID NO: 73)
EQYVDFSLDPTFSIE;

and/or one or more of the following polypeptide sequences from NS5b:

(SEQ ID NO: 74)
LRHHNLVYSTTSRSACQRQK;
(SEQ ID NO: 75)
KVTFDRLQVLDSHYQDVLKE;
(SEQ ID NO: 76)
SVWKDLLEDNVTPIDTTIMA;
(SEQ ID NO: 77)
KGGRKPARLIVFPDLGVRVC;
(SEQ ID NO: 78)
KPARLIVFPDLGVRVCEK;
(SEQ ID NO: 79)
KLPLAVMGSSYGFQYSPGQR;
(SEQ ID NO: 80)
VEFLVQAWKSKKTPMGFSYD;
(SEQ ID NO: 81)
SDIRTEEAIYQCCDLDPQAR;
(SEQ ID NO: 82)
QCCDLDPQARVAIKSLTERL;
(SEQ ID NO: 83)
GYRRCRASGVLT.

The ability of a bacterium of choice to express and secrete the recombinant antigen(s) can be estimated by hydropathy plot and/or directly measured by Western blot analysis as described hereinafter.

In certain embodiments, HCV antigens are chosen to have no region of hydrophobicity that exceeds the peak hydrophobicity of Listeria ActA protein or a fragment thereof used as part of a fusion construct with the HCV antigen(s) of interest. Most preferably, HCV antigens are chosen to have no region of hydrophobicity that exceeds the peak hydrophobicity of Listeria ActA-N100.

Direct detetection of expression of the recombinant antigen in the Western blot may be performed using an antibody that detects an HCV antigen sequence being recombinantly producted, or using an antibody that detects a non-CHV sequence (a “tag”) which is expressed with the HCV antigen as a fusion protein. In examples described hereinafter, the antigen(s) are expressed as fusions with an N-terminal portion of the Listeria ActA protein, and an anti-ActA antibody raised against a synthetic peptide (ATDSEDSSLNTDEWEEEK (SEQ ID NO:24)) corresponding to the mature N terminal 18 amino acids of ActA can be used to detect the expressed protein product.

Assays for testing the immunogenicity of antigens are described herein and are well known in the art. As an example, an antigen recombinantly produced by a bacterium of choice can be optionally constructed to contain the nucleotide sequence encoding an eight amino SIINFEKL (SEQ ID NO:25) peptide (also known as SL8 and ovalbumin₂₅₇₋₂₆₄), positioned in-frame at the carboxyl terminus of the antigen. Compositions such as the C-terminal SL8 epitope serve as a surrogate (i) to demonstrate that the recombinant antigen is being expressed in its entirety from N-terminal to C-terminal, and (ii) to demonstrate the ability of antigen presenting cells to present the recombinant antigen via the MHC class I pathway, using an in vitro antigen presentation assay. Such a presentation assay can be performed using the cloned C57BL/6-derived dendritic cell line DC2.4 together with the B3Z T cell hybridoma cell line as described hereinafter.

Alternatively, or in addition, immunogenicity may be tested using an ELISPOT assay as described hereinafter. ELISPOT assays were originally developed to enumerate B cells secreting antigen-specific antibodies, but have subsequently been adapted for various tasks, especially the identification and enumeration of cytokine-producing cells at the single cell level. Spleens may be harvested from animals inoculated with an appropriate bacterial vaccine, and the isolated splenocytes incubated overnight with or without peptides derived from the one or more HCV antigens expressed by the bacterial vaccine. An immobilized antibody captures any secreted IFN-γ, thus permitting subsequent measurement of secreted IFN-γ, and assessment of the immune response to the vaccine.

### 3. Bacterial Expression Systems

### The “Vaccine Platform”

Selection of a vaccine platform for delivery of the consensus sequence antigens is another critical component for an effective vaccine. A number of bacterial species have been developed for use as vaccines and can be used in the present invention, including, but not limited to, Shigella flexneri, Escherichia coli, Listeria monocytogenes, Yersinia enterocolitica, Salmonella typhimurium, Salmonella typhi or mycobacterium species. This list is not meant to be limiting. See, e.g., WO04/006837; WO07/103,225; and WO07/117,371, each of which is hereby incorporated by reference in its entirety, including all tables, figures, and claims. The bacterial vector used in the vaccine composition may be a facultative, intracellular bacterial vector. The bacterium may be used to deliver a polypeptide described herein to antigen-presenting cells in the host organism. As described herein, L. monocytogenes provides a preferred vaccine platform for expression of the HCV antigen(s).

Both attenuated and commensal microorganisms have been successfully used as carriers for vaccine antigens, but bacterial carriers for the HCV antigens or derivatives thereof are optionally attenuated or killed but metabolically active (KBMA). The genetic background of the carrier strain used in the formulation, the type of mutation selected to achieve attenuation, and the intrinsic properties of the immunogen can be adjusted to optimize the extent and quality of the immune response elicited. The general factors to be considered to optimize the immune response stimulated by the bacterial carrier include: selection of the carrier; the specific background strain, the attenuating mutation and the level of attenuation; the stabilization of the attenuated phenotype and the establishment of the optimal dosage. Other antigen-related factors to consider include: intrinsic properties of the antigen; the expression system, antigen-display form and stabilization of the recombinant phenotype; co-expression of modulating molecules and vaccination schedules.

A preferred feature of the vaccine platform is the ability to initiate both the innate immune response as well as an antigen-specific T cell response against the recombinantly expressed HCV antige(s). For example, L. monocytogenes expressing the HCV antigen(s) described herein induce intrahepatic Type 1 interferon (IFN-α/β) and a downstream cascade of chemokines and cytokines. In response to this intrahepatic immune stimulation, NK cells and antigen presenting cells (APCs) are recruited to the liver. These cells are activated to initiate a T cell response to eradicate Lm; simultaneously a T cell response against the HCV antigens expressed by the L. monocytogenes vaccine platform is also mounted. In certain embodiments, the vaccine platform of the present invention induces an increase at 24 hours following delivery of the vaccine platform to the subject in the serum concentration of one or more, and preferably all, cytokines and chemokines selected from the group consisting of IL-12p70, IFN-γ, IL-6, TNF α, and MCP-1; and induces a CD4+ and/or CD8+ antigen-specific T cell response against one or more HCV antigens expressed by the vaccine platform. In other embodiments, the vaccine platform of the present invention also induces the maturation of resident immature liver NK cells as demonstrated by the upregulation of activation cytolytic activity measured using Cr-labeled YAC-1 cells that were used as target cells.

In various embodiments, the vaccines and immunogenic compositions of the present invention can comprise Listeria monocytogenes configured to express the desired HCV antigen(s). The ability of L. monocytogenes to serve as a vaccine vector has been reviewed in Wesikirch, et al., Immunol. Rev. 158:159-169 (1997). A number of desirable features of the natural biology of L. monocytogenes make it an attractive platform for application to an HCV therapeutic vaccine. The central rationale is that the intracellular lifecycle of L. monocytogenes enables effective stimulation of CD4+ and CD8+ T cell immunity, known to be required for resolution of HCV infection. Multiple pathogen associated molecular pattern (PAMP) receptors including TLRs (TLR2, TLR5, TLR9) and nucleotide-binding oligomerization domains (NOD) are triggered in response to interaction with L. monocytogenes macromolecules upon infection, resulting in the pan-activation of innate immune effectors and release of Th-1 polarizing cytokines, exerting a profound impact on the development of a CD4+ and CD8+ T cell response against the HCV consensus sequence antigens. Lm is particularly well-suited for an HCV vaccine because of its tropism for liver-resident APCs that leads to a potent intrahepatic immune response.

Strains of L. monocytogenes have recently been developed as effective intracellular delivery vehicles of heterologous proteins providing delivery of antigens to the immune system to induce an immune response to clinical conditions that do not permit injection of the disease-causing agent, such as cancer and HIV. See, e.g., U.S. Pat. No. 6,051,237; Gunn et al., J. Immunol., 167:6471-6479 (2001); Liau, et al., Cancer Research, 62: 2287-2293 (2002); U.S. Pat. No. 6,099,848; WO 99/25376; WO 96/14087; and U.S. Pat. No. 5,830,702), each of which is hereby incorporated by reference in its entirety, including all tables, figures, and claims. A recombinant L. monocytogenes vaccine expressing an lymphocytic choriomeningitis virus (LCMV) antigen has also been shown to induce protective cell-mediated immunity to the antigen (Shen et al., Proc. Natl. Acad. Sci. USA, 92: 3987-3991 (1995).

Attenuated and killed but metabolically active forms of L. monocytogenes useful in immunogenic compositions have been produced. WO07/103,225; and WO07/117,371), each of which is hereby incorporated by reference in its entirety, including all tables, figures, and claims. The ActA protein of L. monocytogenes is sufficient to promote the actin recruitment and polymerization events responsible for intracellular movement. A human safety study has reported that oral administration of an actA/plcB-deleted attenuated form of L. monocytogenes caused no serious sequelae in adults (Angelakopoulos et al., Infection and Immunity, 70:3592-3601 (2002)). Other types of attenuated forms of L. monocytogenes have also been described (see, for example, WO 99/25376 and U.S. Pat. No. 6,099,848, which describe auxotrophic, attenuated strains of Listeria that express heterologous antigens).

In certain embodiments, the L. monocytogenes used in the vaccine compositions of the present invention is a live-attenuated strain which comprises an attenuating mutation in actA and/or inlB, and preferably a deletion of all or a portion of actA and inlB (referred to herein as “Lm ΔactA/ΔinlB”), and contains recombinant DNA encoding for the expression of the HCV antigen(s) of interest. These antigen(s) most preferably comprise one or more immunogenic sequences obtained or derived from one or both of the NS5B NS3 consensus sequence antigens. The HCV antigen(s) are preferably under the control of bacterial expression sequences and are stably integrated into the L. monocytogenes genome. Such a L. monocytogenes vaccine strain therefore employs no eukaryotic transcriptional or translational elements.

The invention also contemplates a Listeria attenuated in at least one regulatory factor, e.g., a promoter or a transcription factor. The following concerns promoters. ActA expression is regulated by two different promoters (Lauer, et al. (2002) J. Bacteriol. 184:4177-4186). Together, inlA and inlB expression is regulated by five promoters (Lingnau, et al. (1995) Infect. Immun. 63:3896-3903). The transcription factor prfA is required for transcription of a number of L. monocytogenes genes, e.g., hly, plcA, ActA, mpl, prfA, and iap. PrfA's regulatory properties are mediated by, e.g., the PrfA-dependent promoter (PinlC) and the PrfA-box. The present invention, in certain embodiments, provides a nucleic acid encoding inactivated, mutated, or deleted in at least one of ActA promoter, inlB promoter, PrfA, PinlC, PrfA box, and the like (see, e.g., Lalic Mullthaler, et al. (2001) Mol. Microbiol. 42:111-120; Shetron-Rama, et al. (2003) Mol. Microbiol. 48:1537-1551; Luo, et al. (2004) Mol. Microbiol. 52:39-52). PrfA can be made constitutively active by a Gly145Ser mutation, Gly155Ser mutation, or Glu77Lys mutation (see, e.g., Mueller and Freitag (2005) Infect. Immun. 73:1917-1926; Wong and Freitag (2004) J. Bacteriol. 186:6265-6276; Ripio, et al. (1997) J. Bacteriol. 179:1533-1540).

Attenuation can be effected by, e.g., heat-treatment or chemical modification. Attenuation can also be effected by genetic modification of a nucleic acid that modulates, e.g., metabolism, extracellular growth, or intracellular growth, genetic modification of a nucleic acid encoding a virulence factor, such as listerial prfA, ActA, listeriolysin (LLO), an adhesion mediating factor (e.g., an internalin such as inlA or inlB), mpl, phosphatidylcholine phospholipase C (PC-PLC), phosphatidylinositol-specific phospholipase C (PI PLC; plcA gene), any combination of the above, and the like. Attenuation can be assessed by comparing a biological function of an attenuated Listeria with the corresponding biological function shown by an appropriate parent Listeria.

The present invention, in other embodiments, provides a Listeria that is attenuated by treating with a nucleic acid targeting agent, such as a cross linking agent, a psoralen, a nitrogen mustard, cis platin, a bulky adduct, ultraviolet light, gamma irradiation, any combination thereof, and the like. Typically, the lesion produced by one molecule of cross linking agent involves cross linking of both strands of the double helix. The Listeria of the invention can also be attenuated by mutating at least one nucleic acid repair gene, e.g., uvrA, uvrB, uvrAB, uvrC, uvrD, uvrAB, phrA, and/or a gene mediating recombinational repair, e.g., recA. Moreover, the invention provides a Listeria attenuated by both a nucleic acid targeting agent and by mutating a nucleic acid repair gene. Additionally, the invention encompasses treating with a light sensitive nucleic acid targeting agent, such as a psoralen, and/or a light sensitive nucleic acid cross linking agent, such as psoralen, followed by exposure to ultraviolet light.

Attenuated Listeria useful in the present invention are described in, e.g., in U.S. Pat. Publ. Nos. 2004/0228877 and 2004/0197343, each of which is incorporated by reference herein in its entirety. Various assays for assessing whether a particular strain of Listeria has the desired attenuation are provided, e.g., in U.S. Pat. Publ. Nos. 2004/0228877, 2004/0197343, and 2005/0249748, each of which is incorporated by reference herein in its entirety.

In other embodiments, the L. monocytogenes used in the vaccine compositions of the present invention is a killed but metabolically active (KBMA) platform derived from Lm ΔactA/ΔinlB, and also is deleted of both uvrA and uvrB, genes encoding the DNA repair enzymes of the nucleotide excision repair (NER) pathway, and contains recombinant DNA encoding for the expression of the HCV antigen(s) of interest. These antigen(s) most preferably comprise one or more immunogenic sequences obtained or derived from one or both of the NS5B NS3 consensus sequence antigens. The HCV antigen(s) are preferably under the control of bacterial expression sequences and are stably integrated into the L. monocytogenes genome. The KBMA platform is exquisitely sensitive to photochemical inactivation by the combined treatment with the synthetic psoralen, S-59, and long-wave UV light. While killed, KBMA Lm vaccines can transiently express their gene products, allowing them to escape the phagolysosome and induce functional cellular immunity and protection against wild-typeWT Lm and vaccinia virus challenge.

In certain embodiments, an attenuated or KBMA L. monocytogenes vaccine strain comprise a constitutively active PrfA gene (referred to herein as PrfA* mutants). PrfA is a transcription factor activated intracellularly which induces expression of virulence genes and encoded heterologous antigens (Ags) in appropriately engineered vaccine strains. As noted above, expression of the ActA gene is responsive to prfA, and the ActA promoter is a prfA responsive regulatory element. Inclusion of a prfA G155S allele can confer significant enhanced vaccine potency of live-attenuated or KBMA vaccines. Preferred PrfA mutants are described in U.S. Provisional patent application 61/054,454, entitled COMPOSITIONS COMPRISING PRFA* MUTANT LISTERIA AND METHODS OF USE THEREOF, filed May 19, 2008, which is hereby incorporated in its entirety including all tables, figures, and claims.

The sequence of L. monocytogenes PrfA, which includes a glycine at residue 155, is as follows (SEQ ID NO: 26):

| | |
|---|---|
| MNAQAEEFKK YLETNGIKPK QFHKKELIFN QWDPQEYCIF LYDGITKLTS | 50 |
| ISENGTIMNL QYYKGAFVIM SGFIDTETSV GYYNLEVISE QATAYVIKIN | 100 |
| ELKELLSKNL THFFYVFQTL QKQVSYSLAK FNDFSINGKL GSICGQLLIL | 150 |
| TYVYGKETPD GIKITLDNLT MQELGYSSGI AHSSAVSRII SKLKQEKVIV | 200 |
| YKNSCFYVQN LDYLKRYAPK LDEWFYLACP ATWGKLN | 237 |

The sequence of L. monocytogenes PrfA*, which includes a serine at residue 155, is as follows (SEQ ID NO: 27):

| | |
|---|---|
| MNAQAEEFKK YLETNGIKPK QFHKKELIFN QWDPQEYCIF LYDGITKLTS | 50 |
| ISENGTIMNL QYYKGAFVIM SGFIDTETSV GYYNLEVISE QATAYVIKIN | 100 |
| ELKELLSKNL THFFYVFQTL QKQVSYSLAK FNDFSINGKL GSICGQLLIL | 150 |
| TYVYSKETPD GIKITLDNLT MQELGYSSGI AHSSAVSRII SKLKQEKVIV | 200 |
| YKNSCFYVQN LDYLKRYAPK LDEWFYLACP ATWGKLN | 237 |

### 4. Antigenic Constructs

The antigenic construct expressed by the bacterial vaccine strain of the present invention comprises at a minimum a nucleic acid encoding a secretory sequence operable within the bacterial vaccine platform to support secretion, fused to the HCV antigen(s) to be expressed, wherein the resulting fusion protein is operably linked to regulatory sequences (e.g., a promoter) necessary for expression of the fusion protein by the bacterial vaccine platform. The present invention is not to be limited to polypeptide and peptide antigens that are secreted, but also embraces polypeptides and peptides that are not secreted or cannot be secreted from a Listeria or other bacterium. But preferably, the HCV antigen(s) are expressed in a soluble, secreted form by the bacterial vaccine strain when the strain is inoculated into a recipient.

Table 1 discloses a number of non-limiting examples of signal peptides for use in fusing with a fusion protein partner sequence such as a heterologous antigen. Signal peptides tend to contain three domains: a positively charged N-terminus (1-5 residues long); a central hydrophobic comain (7-15 residues long); and a neutral but polar C-terminal domain.

TABLE 1
Bacterial signal pathway. Signal peptides are identified
by the signal peptidase site.

| | | |
|---|---|---|
| Signal peptidase site | | |
| (cleavage site | | |
| represented by ’) | Gene | Genus/species |

secA1 pathway

| | | |
|---|---|---|
| TEA’KD | hly (LLO) | Listeria monocytogenes |
| (SEQ ID NO: 28) | | |
| VYA’DT | Usp45 | Lactococcus lactis |
| (SEQ ID NO: 29) | | |
| IQA’EV | pag | Bacillus anthracis |
| (SEQ ID NO: 30) | (protective antigen) | |

secA2 pathway

| | | |
|---|---|---|
| ASA’ST | iap | Listeria monocytogenes |
| (SEQ ID NO: 31) | (invasion-associated | |
| | protein) p60 | |
| VGA’FG | NamA lmo2691 | Listeria monocytogenes |
| (SEQ ID NO: 32) | (autolysin) | |
| AFA’ED | * BA_0281 | Bacillus anthracis |
| (SEQ ID NO: 33) | (NLP/P60 Family) | |
| VQA’AE | * atl | Staphylococcus aureus |
| (SEQ ID NO: 34) | (autolysin) | |

Tat pathway

| | | |
|---|---|---|
| DKA’LT | lmo0367 | Listeria monocytogenes |
| (SEQ ID NO: 35) | | |
| VGA’FG | PhoD | Bacillus subtillis |
| (SEQ ID NO: 36) | (alkaline phosphatase) | |

* Bacterial autolysins secreted by sec pathway (not determined whether secA1 or secA2).
Secretory sequences are encompassed by the indicated nucleic acids encoded by the Listeria EGD genome (GenBank Acc. No. NC_003210) at, e.g., nucleotides 45434-456936 (in1A); nucleotides 457021-457125 (in1B); nucleotides 1860200-1860295 (in1C); nucleotides 286219-287718 (in1E); nucleotides 205819-205893 (hly gene; LLO) (see also GenBank Acc. No. P13128); nucleotides 209470-209556 (ActA) (see also GenBank Acc. No. S20887).
The referenced nucleic acid sequences, and corresponding translated amino acid sequences, and the cited amino acid sequences, and the corresponding nucleic acid sequences associated with or cited in that reference, are incorporated by reference herein in their entirety.

In certain exemplary embodiments described hereinafter, the HCV epitope sequence(s) may be expressed as a single polypeptide fused to an amino-terminal portion of the L. monocytogenes ActA protein which permits expression and secretion of HCV fusion protein from the bacterium within the vaccinated host. In these embodiments, the antigenic construct may be a polynucleotide comprising a promoter operably linked to a nucleic acid sequence encoding a fusion protein, wherein the fusion protein comprises (a) modified ActA and (b) one or more HCV epitopes to be expressed as a fusion protein following the modified ActA sequence.

By “modified ActA” is meant a contiguous portion of the L. monocytogenes ActA protein which comprises at least the ActA signal sequence, but does not comprise the entirety of the ActA sequence, or that has at least about 80% sequence identity, at least about 85% sequence identity, at least about 90% sequence identity, at least about 95% sequence identity, or at least about 98% sequence identity to such an ActA sequence. The ActA signal sequence is MGLNRFMRAMMVVFITANCITINPDIIFA (SEQ ID NO: 41). In some embodiments, the promoter is ActA promoter from WO07/103,225; and WO07/117,371, each of which is incorporated by reference in its entirety herein.

By way of example, the modified ActA may comprise at least the first 59 amino acids of ActA, or a sequence having at least about 80% sequence identity, at least about 85% sequence identity, at least about 90% sequence identity, at least about 95% sequence identity, or at least about 98% sequence identity to at least the first 59 amino acids of ActA. In some embodiments, the modified ActA comprises at least the first 100 amino acids of ActA, or a sequence having at least about 80% sequence identity, at least about 85% sequence identity, at least about 90% sequence identity, at least about 95% sequence identity, or at least about 98% sequence identity to the first 100 amino acids of ActA. In other words, in some embodiments, the modified ActA sequence corresponds to an N-terminal fragment of ActA (including the ActA signal sequence) that is truncated at residue 100 or thereafter.

ActA-N100 has the following sequence (SEQ ID NO:37):

| | |
|---|---|
| VGLNRFMRAM MVVFITANCI TINPDIIFAA TDSEDSSLNT DEWEEEKTEE | 50 |
| QPSEVNTGPR YETAREVSSR DIEELEKSNK VKNTNKADLI AMLKAKAEKG | 100 |

In this sequence, the first residue is depicted as a valine; the polypeptide is synthesized by Listeria with a methionine in this position. Thus, ActA-N100 may also have the following sequence (SEQ ID NO:38):

| | |
|---|---|
| MGLNRFMRAM MVVFITANCI TINPDIIFAA TDSEDSSLNT DEWEEEKTEE | 50 |
| QPSEVNTGPR YETAREVSSR DIEELEKSNK VKNTNKADLI AMLKAKAEKG | 100 |

ActA-N100 may also comprise one or more additional residues lying between the C-terminal residue of the modified ActA and the HCV antigen sequence. In the following sequences, ActA-N100 is extended by two residues added by inclusion of a BamH1 site:

| (SEQ ID NO: 39) | |
|---|---|
| VGLNRFMRAM MVVFITANCI TINPDIIFAA TDSEDSSLNT DEWEEEKTEE | 50 |
| QPSEVNTGPR YETAREVSSR DIEELEKSNK VKNTNKADLI AMLKAKAEKG | 100 |
| GS, | 102 |

which when synthesized with a first residue methionine has the sequence:

| (SEQ ID NO: 40) | |
|---|---|
| MGLNRFMRAM MVVFITANCI TINPDIIFAA TDSEDSSLNT DEWEEEKTEE | 50 |
| QPSEVNTGPR YETAREVSSR DIEELEKSNK VKNTNKADLI AMLKAKAEKG | 100 |
| GS. | 102 |

Exemplary constructs are described hereinafter and in WO07/103,225, which is incorporated by reference herein. ANZ-100 (formerly known as CRS-100; BB-IND 12884 and clinicaltrials.gov identifier NCT00327652) consists of a L. monocytogenes ΔactA/ΔinlB platform without any exogenous antigen expression sequences. In the exemplary constructs described in WO07/103,225, this platform has been engineered to express human Mesothelin as a fusion with ActA-N100. The mesothelin expression vaccine has been evaluated in subjects with advanced carcinoma with liver metastases using CRS-207 (BB-IND 13389 and clinicaltrials.gov identifier NCT00585845) which is currently being evaluated in a Phase 1 trial in subjects with advanced carcinomas that are known to over-express mesothelin. The present invention contemplates modification of this vaccine by replacing the mesothelin sequences with an HCV antigen sequence.

As sequences encoded by one organism are not necessarily codon optimized for optimal expression in a chosen vaccine platform bacterial strain, the present invention also provides nucleic acids that are altered by codon optimized for expressing by a bacterium such as L. monocytogenes.

In various embodiments, at least one percent of any non-optimal codons are changed to provide optimal codons, more normally at least five percent are changed, most normally at least ten percent are changed, often at least 20% are changed, more often at least 30% are changed, most often at least 40%, usually at least 50% are changed, more usually at least 60% are changed, most usually at least 70% are changed, optimally at least 80% are changed, more optimally at least 90% are changed, most optimally at least 95% are changed, and conventionally 100% of any non-optimal codons are codon-optimized for Listeria expression (Table 2).

TABLE 2
Optimal codons for expression in Listeria.

| Amino Acid | A | R | N | D | C | Q | E | G | H | I |
|---|---|---|---|---|---|---|---|---|---|---|
| Optimal | GCA | CGU | AAU | GAU | UGU | CAA | GAA | GGU | CAU | AUU |
| Listeria codon | | | | | | | | | | |
| Amino Acid | L | K | M | F | P | S | T | W | Y | V |
| Optimal | UUA | AAA | AUG | UUU | CCA | AGU | ACA | UGG | UAU | GUU |
| Listeria codon | | | | | | | | | | |

The invention supplies a number of listerial species and strains for making or engineering a vaccine platform of the present invention. The Listeria of the present invention is not to be limited by the species and strains disclosed in Table 3.

TABLE 3
Strains of Listeria suitable for use in the present invention,
e.g., as a vaccine or as a source of nucleic acids.

| | |
|---|---|
| L. monocytogenes 10403S wild type. | Bishop and Hinrichs (1987) J. Immunol. |
| | 139: 2005-2009; Lauer, et al. (2002) J. Bact. |
| | 184: 4177-4186. |
| L. monocytogenes DP-L4056 (phage cured). | Lauer, et al. (2002) J. Bact. 184: 4177-4186. |
| The prophage-cured 10403S strain is designated | |
| DP-L4056. | |
| L. monocytogenes DP-L4027, which is | Lauer, et al. (2002) J. Bact. 184: 4177-4186; |
| DP-L2161, phage cured, deleted in hly gene. | Jones and Portnoy (1994) Infect. Immunity |
| | 65: 5608-5613. |
| L. monocytogenes DP-L4029, which is DP- | Lauer, et al. (2002) J. Bact. 184: 4177-4186; |
| L3078, phage cured, deleted in ActA. | Skoble, et al. (2000) J. Cell Biol. 150: 527- |
| | 538. |
| L. monocytogenes DP-L4042 (delta PEST) | Brockstedt, et al. (2004) Proc. Natl. Acad. |
| | Sci. USA 101: 13832-13837; supporting |
| | information. |
| L. monocytogenes DP-L4097 (LLO-S44A). | Brockstedt, et al. (2004) Proc. Natl. Acad. |
| | Sci. USA 101: 13832-13837; supporting |
| | information. |
| L. monocytogenes DP-L4364 (delta lplA; | Brockstedt, et al. (2004) Proc. Natl. Acad. |
| lipoate protein ligase). | Sci. USA 101: 13832-13837; supporting |
| | information. |
| L. monocytogenes DP-L4405 (delta inlA). | Brockstedt, et al. (2004) Proc. Natl. Acad. |
| | Sci. USA 101: 13832-13837; supporting |
| | information. |
| L. monocytogenes DP-L4406 (delta inlB). | Brockstedt, et al. (2004) Proc. Natl. Acad. |
| | Sci. USA 101: 13832-13837; supporting |
| | information. |
| L. monocytogenes CS-L0001 (delta ActA-delta | Brockstedt, et al. (2004) Proc. Natl. Acad. |
| inlB). | Sci. USA 101: 13832-13837; supporting |
| | information. |
| L. monocytogenes CS-L0002 (delta ActA-delta | Brockstedt, et al. (2004) Proc. Natl. Acad. |
| lplA). | Sci. USA 101: 13832-13837; supporting |
| | information. |
| L. monocytogenes CS-L0003 (L461T-delta | Brockstedt, et al. (2004) Proc. Natl. Acad. |
| lplA). | Sci. USA 101: 13832-13837; supporting |
| | information. |
| L. monocytogenes DP-L4038 (delta ActA-LLO | Brockstedt, et al. (2004) Proc. Natl. Acad. |
| L461T). | Sci. USA 101: 13832-13837; supporting |
| | information. |
| L. monocytogenes DP-L4384 (S44A-LLO | Brockstedt, et al. (2004) Proc. Natl. Acad. |
| L461T). | Sci. USA 101: 13832-13837; supporting |
| | information. |
| L. monocytogenes. Mutation in lipoate protein | O'Riordan, et al. (2003) Science 302: 462- |
| ligase (LplA1). | 464. |
| L. monocytogenes DP-L4017 (10403S | U.S. Provisional Pat. application Ser. No. |
| hly (L461T) point mutation in hemolysin gene. | 60/490,089 filed Jul. 24, 2003. |
| L. monocytogenes EGD. | GenBank Acc. No. AL591824. |
| L. monocytogenes EGD-e. | GenBank Acc. No. NC_003210. ATCC |
| | Acc. No. BAA-679. |
| L. monocytogenes strain EGD, complete | GenBank Acc. No. AL591975 |
| genome, segment 3/12 | |
| L. monocytogenes. | ATCC Nos. 13932; 15313; 19111-19120; |
| | 43248-43251; 51772-51782. |
| L. monocytogenes DP-L4029 deleted in uvrAB. | U.S. Provisional Pat. application Ser. No. |
| | 60/541,515 filed Feb. 2, 2004; U.S. |
| | Provisional Pat. application Ser. No. 60/490,080 |
| | filed Jul. 24, 2003. |
| L. monocytogenes DP-L4029 deleted in uvrAB | U.S. Provisional Pat. application Ser. No. |
| treated with a psoralen. | 60/541,515 filed Feb. 2, 2004. |
| L. monocytogenes ActA-/inlB- double mutant. | Deposited with ATCC on Oct. 3, 2003. |
| | Acc. No. PTA-5562. |
| L. monocytogenes lplA mutant or hly mutant. | U.S. Pat. application No. 20040013690 of |
| | Portnoy, et al. |
| L. monocytogenes DAL/DAT double mutant. | U.S. Pat. application No. 20050048081 of |
| | Frankel and Portnoy. |
| L. monocytogenes str. 4b F2365. | GenBank Acc. No. NC_002973. |
| Listeria ivanovii | ATCC No. 49954 |
| Listeria innocua Clip11262. | GenBank Acc. No. NC_003212; AL592022. |
| Listeria innocua, a naturally occurring | Johnson, et al. (2004) Appl. Environ. |
| hemolytic strain containing the PrfA-regulated | Microbiol. 70: 4256-4266. |
| virulence gene cluster. | |
| Listeria seeligeri. | Howard, et al. (1992) Appl. Eviron. |
| | Microbiol. 58: 709-712. |
| Listeria innocua with L. monocytogenes | Johnson, et al. (2004) Appl. Environ. |
| pathogenicity island genes. | Microbiol. 70: 4256-4266. |
| Listeria innocua with L. monocytogenes | See, e.g., Lingnau, et al. (1995) Infection |
| internalin A gene, e.g., as a plasmid or as a | Immunity 63: 3896-3903; Gaillard, et al. |
| genomic nucleic acid. | (1991) Cell 65: 1127-1141). |

The present invention encompasses reagents and methods that comprise the above listerial strains, as well as these strains that are modified, e.g., by a plasmid and/or by genomic integration, to contain a nucleic acid encoding one of, or any combination of, the following genes: hly (LLO; listeriolysin); iap (p60); inlA; inlB; inlC; dal (alanine racemase); daaA (dat; D-amino acid aminotransferase); plcA; plcB; ActA; or any nucleic acid that mediates growth, spread, breakdown of a single walled vesicle, breakdown of a double walled vesicle, binding to a host cell, uptake by a host cell. The present invention is not to be limited by the particular strains disclosed above.

### 4. Therapeutic Compositions

The vaccine compositions described herein can be administered to a host, either alone or in combination with a pharmaceutically acceptable excipient, in an amount sufficient to induce an appropriate immune response to HCV infection. The immune response can comprise, without limitation, specific immune response, non specific immune response, both specific and non specific response, innate response, primary immune response, adaptive immunity, secondary immune response, memory immune response, immune cell activation, immune cell proliferation, immune cell differentiation, and cytokine expression. The vaccines of the present invention can be stored, e.g., frozen, lyophilized, as a suspension, as a cell paste, or complexed with a solid matrix or gel matrix.

In certain embodiments, after the subject has been administered an effective dose of a vaccine containing the immunogenic HCV antigen polypeptides to prime the immune response, a second vaccine is administered. This is referred to in the art as a “prime-boost” regimen. In such a regimen, the compositions and methods of the present invention may be used as the “prime” delivery, as the “boost” delivery, or as both a “prime” and a “boost.”

As an example, a first vaccine comprised of killed but metabolically active Listeria that encodes and expresses the antigen polypeptide(s) may be delivered as the “prime,” and a second vaccine comprised of attenuated but metabolically active Listeria that encodes the antigen polypeptide(s) may be delivered as the “boost.” It should be understood, however, that each of the prime and boost need not utilize the methods and compositions of the present invention. Rather, the present invention contemplates the use of other vaccine modalities together with the bacterial vaccine methods and compositions of the present invention. The following are examples of suitable mixed prime-boost regimens: a DNA (e.g., plasmid) vaccine prime/bacterial vaccine boost; a viral vaccine prime/bacterial vaccine boost; a protein vaccine prime/bacterial vaccine boost; a DNA prime/bacterial vaccine boost plus protein vaccine boost; a bacterial vaccine prime/DNA vaccine boost; a bacterial vaccine prime/viral vaccine boost; a bacterial vaccine prime/protein vaccine boost; a bacterial vaccine prime/bacterial vaccine boost plus protein vaccine boost; etc. This list is not meant to be limiting

The prime vaccine and boost vaccine may be administered by the same route or by different routes. The term “different routes” encompasses, but is not limited to, different sites on the body, for example, a site that is oral, non-oral, enteral, parenteral, rectal, intranode (lymph node), intravenous, arterial, subcutaneous, intramuscular, intratumor, peritumor, intratumor, infusion, mucosal, nasal, in the cerebrospinal space or cerebrospinal fluid, and so on, as well as by different modes, for example, oral, intravenous, and intramuscular.

An effective amount of a prime or boost vaccine may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of the vaccine. Where there is more than one administration of a vaccine or vaccines in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The invention is not limited to dosing intervals that are spaced equally in time, but encompass doses at non-equal intervals, such as a priming schedule consisting of administration at 1 day, 4 days, 7 days, and 25 days, just to provide a non-limiting example.

In certain embodiments, administration of the boost vaccination can be initiated at about 5 days after the prime vaccination is initiated; about 10 days after the prime vaccination is initiated; about 15 days; about 20 days; about 25 days; about 30 days; about 35 days; about 40 days; about 45 days; about 50 days; about 55 days; about 60 days; about 65 days; about 70 days; about 75 days; about 80 days, about 6 months, and about 1 year after administration of the prime vaccination is initiated. Preferably one or both of the prime and boost vaccination comprises delivery of a composition of the present invention.

A “pharmaceutically acceptable excipient” or “diagnostically acceptable excipient” includes but is not limited to, sterile distilled water, saline, phosphate buffered solutions, amino acid based buffers, or bicarbonate buffered solutions. An excipient selected and the amount of excipient used will depend upon the mode of administration. Administration may be oral, intravenous, subcutaneous, dermal, intradermal, intramuscular, mucosal, parenteral, intraorgan, intralesional, intranasal, inhalation, intraocular, intramuscular, intravascular, intranodal, by scarification, rectal, intraperitoneal, or any one or combination of a variety of well-known routes of administration. The administration can comprise an injection, infusion, or a combination thereof.

Administration of the vaccine of the present invention by a non oral route can avoid tolerance. Methods are known in the art for administration intravenously, subcutaneously, intramuscularly, intraperitoneally, orally, mucosally, by way of the urinary tract, by way of a genital tract, by way of the gastrointestinal tract, or by inhalation.

An effective amount for a particular patient may vary depending on factors such as the condition being treated, the overall health of the patient, the route and dose of administration and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The vaccines of the present invention can be administered in a dose, or dosages, where each dose comprises at least 1000 bacterial cells/kg body weight; normally at least 10,000 cells; more normally at least 100,000 cells; most normally at least 1 million cells; often at least 10 million cells; more often at least 100 million cells; typically at least 1 billion cells; usually at least 10 billion cells; conventionally at least 100 billion cells; and sometimes at least 1 trillion cells/kg body weight. The present invention provides the above doses where the units of bacterial administration is colony forming units (CFU), the equivalent of CFU prior to psoralen treatment, or where the units are number of bacterial cells.

The vaccines of the present invention can be administered in a dose, or dosages, where each dose comprises between 10⁷ and 10⁸ bacteria per 70 kg body weight (or per 1.7 square meters surface area; or per 1.5 kg liver weight); 2×10⁷ and 2×10⁸ bacteria per 70 kg body weight (or per 1.7 square meters surface area; or per 1.5 kg liver weight); 5×10⁷ and 5×10⁸ bacteria per 70 kg body weight (or per 1.7 square meters surface area; or per 1.5 kg liver weight); 10⁸ and 10⁹ bacteria per 70 kg body weight (or per 1.7 square meters surface area; or per 1.5 kg liver weight); between 2.0×10⁸ and 2.0×10⁹ bacteria per 70 kg (or per 1.7 square meters surface area, or per 1.5 kg liver weight); between 5.0×10⁸ to 5.0×10⁹ bacteria per 70 kg (or per 1.7 square meters surface area, or per 1.5 kg liver weight); between 10⁹ and 10¹⁰ bacteria per 70 kg (or per 1.7 square meters surface area, or per 1.5 kg liver weight); between 2×10⁹ and 2×10¹° bacteria per 70 kg (or per 1.7 square meters surface area, or per 1.5 kg liver weight); between 5×10⁹ and 5×10¹° bacteria per 70 kg (or per 1.7 square meters surface area, or per 1.5 kg liver weight); between 10¹¹ and 10¹² bacteria per 70 kg (or per 1.7 square meters surface area, or per 1.5 kg liver weight); between 2×10¹¹ and 2×10¹² bacteria per 70 kg (or per 1.7 square meters surface area, or per 1.5 kg liver weight); between 5×10¹¹ and 5×10¹² bacteria per 70 kg (or per 1.7 square meters surface area, or per 1.5 kg liver weight); between 10¹² and 10¹³ bacteria per 70 kg (or per 1.7 square meters surface area); between 2×10¹² and 2×10¹³ bacteria per 70 kg (or per 1.7 square meters surface area, or per 1.5 kg liver weight); between 5×10¹² and 5×10¹³ bacteria per 70 kg (or per 1.7 square meters surface area, or per 1.5 kg liver weight); between 10¹³ and 10¹⁴ bacteria per 70 kg (or per 1.7 square meters surface area, or per 1.5 kg liver weight); between 2×10¹³ and 2×10¹⁴ bacteria per 70 kg (or per 1.7 square meters surface area, or per 1.5 kg liver weight); 5×10¹³ and 5×10¹⁴ bacteria per 70 kg (or per 1.7 square meters surface area, or per 1.5 kg liver weight); between 10¹⁴ and 10¹⁵ bacteria per 70 kg (or per 1.7 square meters surface area, or per 1.5 kg liver weight); between 2×10¹⁴ and 2×10¹⁵ bacteria per 70 kg (or per 1.7 square meters surface area, or per 1.5 kg liver weight); and so on, wet weight.

Also provided is one or more of the above doses, where the dose is administered by way of one injection every day, one injection every two days, one injection every three days, one injection every four days, one injection every five days, one injection every six days, or one injection every seven days, where the injection schedule is maintained for, e.g., one day only, two days, three days, four days, five days, six days, seven days, two weeks, three weeks, four weeks, five weeks, or longer. The invention also embraces combinations of the above doses and schedules, e.g., a relatively large initial bacterialdose, followed by relatively small subsequent doses, or a relatively small initial dose followed by a large dose.

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

The present invention encompasses a method of administering Listeria that is oral. Also provided is a method of administering Listeria that is intravenous. Moreover, what is provided is a method of administering Listeria that is oral, intramuscular, intravenous, intradermal and/or subcutaneous. The invention supplies a Listeria bacterium, or culture or suspension of Listeria bacteria, prepared by growing in a medium that is meat based, or that contains polypeptides derived from a meat or animal product. Also supplied by the present invention is a Listeria bacterium, or culture or suspension of Listeria bacteria, prepared by growing in a medium that does not contain meat or animal products, prepared by growing on a medium that contains vegetable polypeptides, prepared by growing on a medium that is not based on yeast products, or prepared by growing on a medium that contains yeast polypeptides.

Methods for co-administration with an additional therapeutic agent are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., PA; Chabner and Longo (eds.) (2001) Cancer Chemotherapy and Biotherapy, Lippincott, Williams & Wilkins, Phila., PA).

The present invention provides reagents for administering in conjunction with a vaccine composition of the present invention. These reagents include other HCV therapeutics, including IFN-α, ribavirin, levovirin, viramidine, telaprevir, boceprevir, PEG-IFN-α, and other immunotherapeutics. This list is not meant to be limiting. The reagents can be administered simultaneously with or independently (before or after) from the vaccine composition of the present invention. For example, the reagent can be administered immediately before (or after) the vaccine composition of the present invention, on the same day as, one day before (or after), one week before (or after), one month before (or after), or two months before (or after) the vaccine composition of the present invention, and the like.

Additional agents which are beneficial to raising a cytolytic T cell response may be used as well. Such agents are termed herein carriers. These include, without limitation, B7 costimulatory molecule, interleukin-2, interferon-γ, GM-CSF, CTLA-4 antagonists, OX-40/OX-40 ligand, CD40/CD40 ligand, sargramostim, levamisol, vaccinia virus, Bacille Calmette-Guerin (BCG), liposomes, alum, Freund's complete or incomplete adjuvant, detoxified endotoxins, mineral oils, surface active substances such as lipolecithin, pluronic polyols, polyanions, peptides, and oil or hydrocarbon emulsions. Carriers for inducing a T cell immune response which preferentially stimulate a cytolytic T cell response versus an antibody response are preferred, although those that stimulate both types of response can be used as well. In cases where the agent is a polypeptide, the polypeptide itself or a polynucleotide encoding the polypeptide can be administered. The carrier can be a cell, such as an antigen presenting cell (APC) or a dendritic cell. Antigen presenting cells include such cell types aas macrophages, dendritic cells and B cells. Other professional antigen-presenting cells include monocytes, marginal zone Kupffer cells, microglia, Langerhans' cells, interdigitating dendritic cells, follicular dendritic cells, and T cells. Facultative antigen-presenting cells can also be used. Examples of facultative antigen-presenting cells include astrocytes, follicular cells, endothelium and fibroblasts. The carrier can be a bacterial cell that is transformed to express the polypeptide or to deliver a polynucleoteide which is subsequently expressed in cells of the vaccinated individual. Adjuvants, such as aluminum hydroxide or aluminum phosphate, can be added to increase the ability of the vaccine to trigger, enhance, or prolong an immune response. Additional materials, such as cytokines, chemokines, and bacterial nucleic acid sequences, like CpG, a toll-like receptor (TLR) 9 agonist as well as additional agonists for TLR 2, TLR 4, TLR 5, TLR 7, TLR 8, TLR9, including lipoprotein, LPS, monophosphoryl lipid A, lipoteichoic acid, imiquimod, resiquimod, and other like immune modulators used separately or in combination with the described compositions are also potential adjuvants. Other representative examples of adjuvants include the synthetic adjuvant QS-21 comprising a homogeneous saponin purified from the bark of Quillaja saponaria and Corynebacterium parvum (McCune et al., Cancer, 1979; 43:1619). It will be understood that the adjuvant is subject to optimization. In other words, the skilled artisan can engage in routine experimentation to determine the best adjuvant to use.

An effective amount of a therapeutic agent is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

The reagents and methods of the present invention provide a vaccine comprising only one vaccination; or comprising a first vaccination; or comprising at least one booster vaccination; at least two booster vaccinations; or at least three booster vaccinations. Guidance in parameters for booster vaccinations is available. See, e.g., Marth (1997) Biologicals 25:199-203; Ramsay, et al. (1997) Immunol. Cell Biol. 75:382-388; Gherardi, et al. (2001) Histol. Histopathol. 16:655-667; Leroux-Roels, et al. (2001) ActA Clin. Belg. 56:209-219; Greiner, et al. (2002) Cancer Res. 62:6944-6951; Smith, et al. (2003) J. Med. Virol. 70:Suppl.1:S38-541; Sepulveda-Amor, et al. (2002) Vaccine 20:2790-2795).

Formulations of therapeutic agents may be prepared for storage by mixing with physiologically acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

### EXAMPLES

The following examples serve to illustrate the present invention. These examples are in no way intended to limit the scope of the invention.

### Example 1

### Development of ANZ 100

The L. monocytogenes ANZ 100 vaccine platform strain was derived from the L. monocytogenes strain DP L4056, a prophage-free derivative of L. monocytogenes strain 10403S, which itself is a streptomycin-resistant variant of the wild-type L. monocytogenes strain 10403. Strain Lm 10403 was first isolated from human skin lesions (Edman 1968), and the streptomycin-resistant strain 10403S was first described by Bishop and Hinrichs (Bishop 1987). Streptomycin resistance in 10403S has been mapped to a single mutation at codon 56 of the ribosomal protein gene rpsL in which a T to C nucleic acid substitution results in insertion of an R (Lys) instead of K (t(Arg) amino acid at position 56, and the process used to isolate strain DP L4056 from Lm 10403S has been previously described in detail (Lauer 2002).

Removal of the actA and inlB virulence genes was accomplished by homologous recombination. The deletion of each gene required three steps: (1) construction of the recombination plasmid containing the deletion allele, (2) integration of the plasmid into the host chromosome, and (3) excision of plasmid vector sequence and the wild-type allele. The Lm ΔactA/ΔinlB strain CERS 382.20 was selected from two PCR-positive candidates based on virulence and immunogenicity in mice. Virulence in C57Bl/6 mice was evaluated using a competitive index assay. Both candidates were equally attenuated, and similar levels of attenuation were observed with a non-phage cured ΔactA/ΔinlB research strain. Next, candidates were tested in Balb/C mice for their ability to prime T-cells specific for an immunodominant Kd-restricted LLO epitope. There were no discernable differences in immunopotency among the candidates or the non-phage cured ΔactA/ΔinlB strain. Since both candidates were comparable to the non-phage cured research strain based on attenuation and immunopotency, one candidate (CERS 382.20) was chosen for further characterization.

Overlapping PCR products encompassing both actA and inlB loci were amplified from fresh colony suspensions of Lm strain CERS 382.20 and DP L4056. PCR products were sequenced, assembled into a single contiguous DNA sequence for each locus, thus confirming precise start- to stop-codon deletions of both actA and inlB genes in CERS 382.20. The rpsL mutation at codon 56 described in 10403S was conserved in CERS 382.20 and documented by sequence analysis of PCR products amplified directly from chromosomal DNA. The streptomycin-resistant phenotype of CERS 382.20 was demonstrated by growth on selective media. The streptomycin-resistant phenotype is used to facilitate identification of the clinical strain and distinguishes it from other Listeria and non-Listeria species.

### Example 2

### Evaluation of HCV Antigens

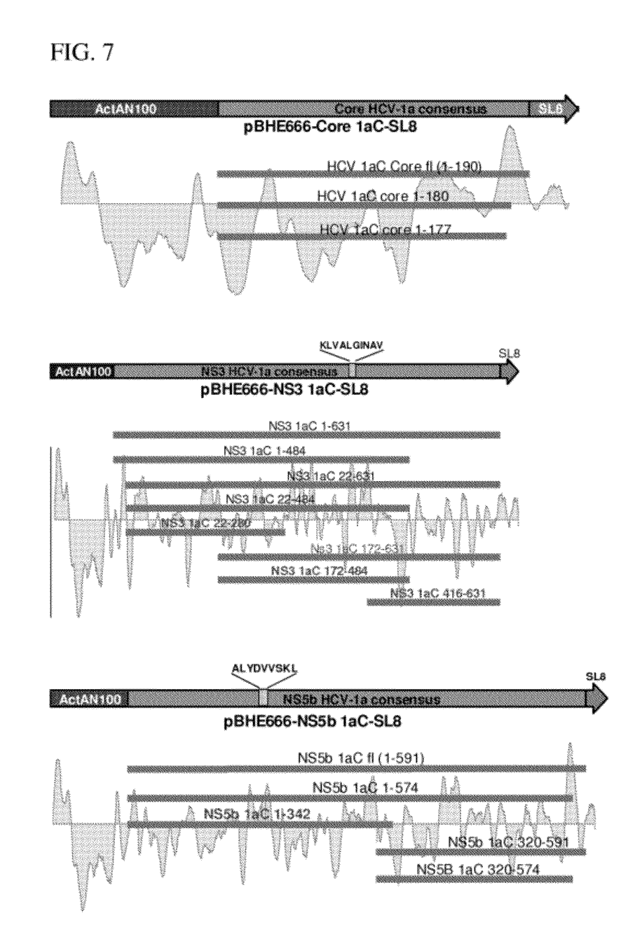

The Kyte-Doolittle hydropathy plot is a widely applied scale for delineating hydrophobic character of a protein. Hydrophobicity is calculated from solvation enthalpy for an individual amino acid residue and summing the values over a sliding window of 5 to 7 amino acids. Regions with values above 0 are hydrophobic in character. An initial Kyte-Doolittle evaluation of HCV core, NS3, and NS5b antigens was to identify regions which are less than or equal to the peak hydrophobic value obtained from ActA-N100. Values greater than this can indicate a polypeptide sequence which does not express well in Listeria. These results are depicted in FIG. 7.

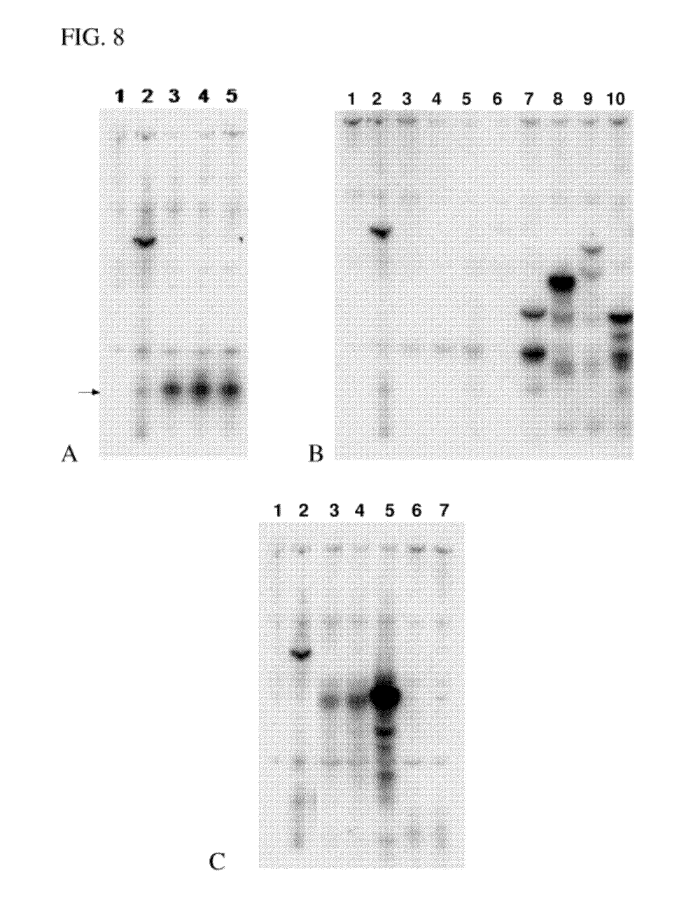

FIG. 8 depicts antigen recombinant expression by Listeria of various ActA-N100 HCV antigen fusions as measured by Western blot. Individual HCV sequences (core sequences 1-190, 1-180, and 1-177; NS3 sequences 1-631, 1-484, 22-631, 22-484, 22-280, 172-484, 172-631, and 416-631; and NS5 sequences 1-574, 1-342, 320-591, and 320-574) were expressed as ActA-N100 fusions from an antigen expression cassette under the control of a bacterial promoter (L. monocytogenes ActA promoter). The expression cassette was stably integrated into the L. monocytogenes genome The L. monocytogenes actA promoter was chosen because it is highly induced in host cells.

Western blots from broth culture were performed on equivalent amounts of TCA-precipitated supernatants of bacterial cultures grown in yeast extract media to an OD₆₀₀ of 0.7 (late log). For western blots from Lm infected host cells, J774 cells or DC2.4 cells were inoculated with an multiplicity of infection (MOI) of 50 or 100 for 1 hour, the cells were washed 3× with PBS and DMEM media supplemented with 50 μg/mL gentamycin. For early timepoints, DC2.4s were harvested at 1.5 or 2.5 hr post infection. For late time points, J774 cells were harvested at 7 hours. Cells were lysed with SDS sample buffer, collected and run on 4-12% polyacrylamide gels and transferred to nitrocellulose membranes for Western blot analysis. All western blots utilized a polyclonal antibody raised against the mature N-terminus of the ActA protein.

In the figure, lanes 1 and 2 in each panel are negative and positive controls showing no antigen insert and mesothelin expression by Listeria strain CRS-207. Panel A shows core sequences 1-190, 1-180, and 1-177 in lanes 3, 4, and 5. Panel B shows NS3 sequences 1-631, 1-484, 22-631, 22-484, 22-280, 172-484, 172-631, and 416-631 in lanes 3-10. Panel C shows NS5 sequences 1-574, 1-342, 320-591, and 320-574 in lanes 3-7. As seen in these figures, NS3₁₇₂₋₄₈₄ exhibits strong expression, as does NS5b₁₋₃₄₂. The arrow in FIG. 8 shows a protein product being produced that is at a substantially lower molecular weight than that predicted from the expressed sequence.

The recombinant expression of these various ActA-N100 HCV antigen fusions from recombinant L. monocytogenes were also used to evaluate the presence of an intact C-terminal SL8 mouse T cell epitope. The SL8 epitope serves as a tag to demonstrate that the recombinant antigen is being expressed in its entirety from N-terminal to C-terminal, and to demonstrate the ability of antigen presenting cells to present the recombinant antigen via the MHC class I pathway. The respective L. monocytogenes was used to infect DC2.4 cells. With infection, the recombinant L. monocytogenes expressed and secreted the fusion polypeptides within the DC2.4 cells. If the DC2.4 cells properly present the peptides, this may be detected by way of a reporter T cell hybridoma line (B3Z T cell hybridoma). Results are presented in the following table:

| Antigen construct | OD₅₉₅ |
|---|---|
| core sequences | |
| 1-190 | 0.27 |
| 1-180 | 0.32 |
| 1-177 | 0.33 |
| NS3 sequences | |
| 1-631 | 0.82 |
| 1-484 | 0.83 |
| 22-631 | 0.83 |
| 22-484 | 0.43 |
| 22-280 | 0.88 |
| 172-484 | 0.88 |
| 172-631 | 0.9 |
| 416-631 | 0.9 |
| NS5 sequences | |
| 1-574 | 0.35 |
| 1-342 | 0.88 |
| 320-591 | 0.18 |
| 320-574 | 0.55 |

Based on Kyte-Doolittle hydropathy analysis, protein expression results, and antigen presentation data, amino acids 1-342 of the NS5B protein and amino acids 172-484 of NS3 were selected for use in a vaccine construct.

### Example 3

### Development of ANZ 521

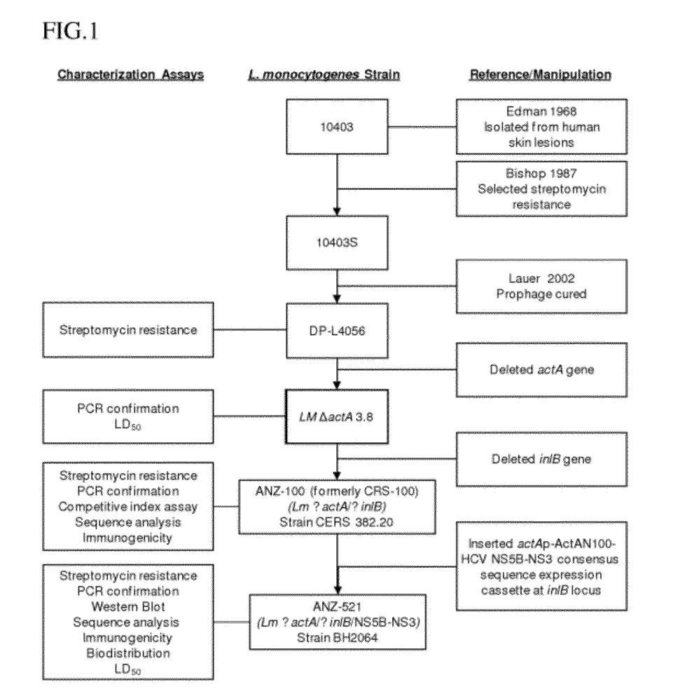

L. monocytogenes strain ANZ 521 is a Listeria vaccine strain based upon the ANZ 100 vaccine platform. A schematic depicting the origins and derivation of ANZ 521 is provided in FIG. 1.

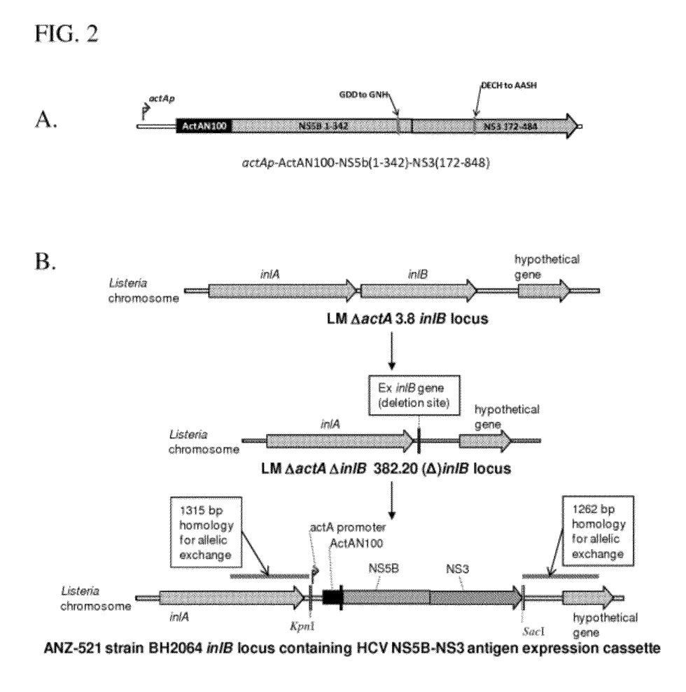

To develop ANZ 521, an antigen expression cassette (FIG. 2A) was constructed that encodes portions of HCV gene products NS5b and NS3 under the control of a bacterial promoter (L. monocytogenes ActA promoter). The expression cassette was stably integrated into the L. monocytogenes genome (FIG. 2B). The L. monocytogenes actA promoter was chosen because it is highly induced in host cells. The HCV antigen comprising NS5b and NS3 sequences is expressed as a single polypeptide fused to the amino-terminal 100 amino acids of the ActA protein (“ActA-N100”), which maximizes expression and secretion of HCV NS5B-NS3 fusion protein from the bacterium within the context of the infected APC in the vaccinated host.

The expressed mature ActA-N100-HCV NS5B-NS3 fusion protein is 730 amino acids in length with a predicted molecular weight of 78.5 kDa. The NS5b-NS3 antigen expression cassette encodes amino acids 1-342 of the NS5b protein (full-length NS5b is 591aa), and amino acids 172-484 of NS3 (full-length NS3 is 631aa). The HCV NS5b-NS3 amino acid sequence is derived from the HCV NS5b and NS3 consensus sequences (Cox 2005, Ray 2005) and the encoding DNA sequence was re-synthesized to utilize optimal codons for expression in L. monocytogenes. Because the antigens are truncated and are synthesized and secreted from L. monocytogenes as a fusion protein, it is unlikely that they would have their native structure or activity, but to ensure that the proteins do not have their endogenous activity, site-specific mutations were engineered into motifs critical for the activity of each protein. To ensure that the NS5B polymerase is non-functional, the amino acid sequence was altered to contain a GDD to GNH (beginning at amino acid 319 of NS5b) inactivating double mutation wherein each change completely inactivates the RNA polymerase activity (Lohmann 1997). To inactivate the NS3 helicase activity, motif II (DECH) was mutated to AASH beginning at amino acid 292 of NS3 (Wardell 1999). NS3 is unlikely to have protease activity because the catalytic serine is not present in this construct (Bartenschlager 1993).

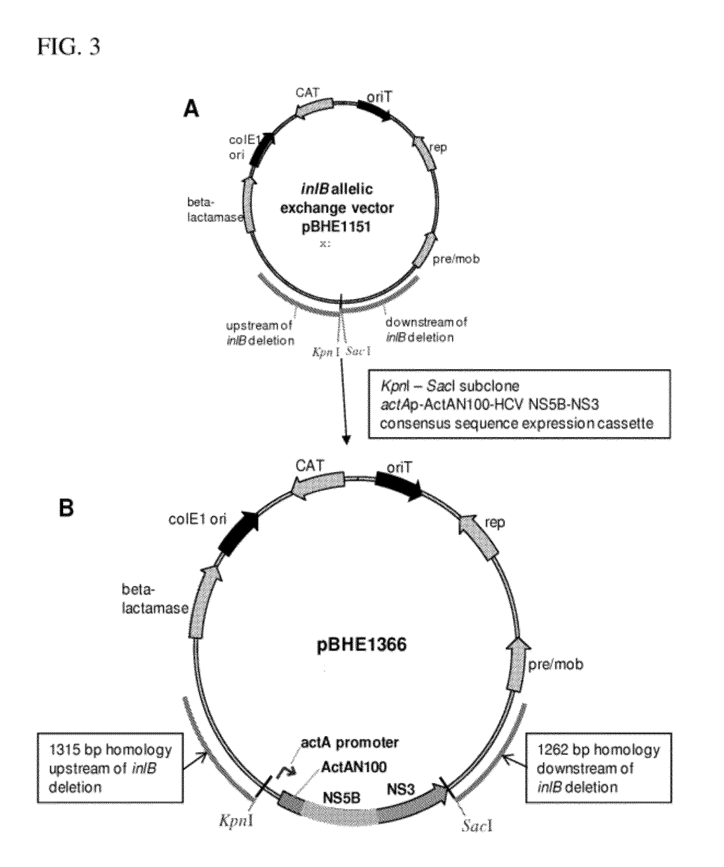

The ActA-N100-HCV NS5B-NS3 consensus sequence antigen expression cassette was inserted at the (Δ)inlB locus of the Lm ΔactA/ΔinlB “parent” strain chromosome using standard allelic exchange techniques. An allelic exchange vector was constructed to direct homologous recombination to the inlB locus of the chromosome of Strain 382.20 (FIG. 3A). First, splicing by overlap extension (SOE) PCR was used to fuse 1315 bp of homology upstream of the inlB locus to 1265 bp of homology downstream of the inlB locus. Unique KpnI and Sad restriction enzyme sites were added at the junction of the upstream and downstream homology, which were used to insert the HCV NS5b-NS3 antigen cassette into the vector. The resulting 2606 bp PCR product was cloned into the temperature sensitive allelic exchange vector pBHE261 which is a derivative of the allelic exchange vector pKSV7 (Smith 1992) that has been modified to contain an origin of transfer (oriT) to facilitate conjugation resulting in the “pBHE1151 inlB allelic exchange vector” plasmid. Next, the expression cassette consisting of the actA promoter, the amino-terminal 300 bp of the actA gene (encoding the ActA-N100 fragtment), and the codon-optimized HCV NS5b-NS3 consensus sequence antigen fusion was cloned into pBHE1151 inlB allelic exchange vector, resulting in the plasmid pBHE1366 (FIG. 3B).

Plasmid pBHE1366 was transferred into into strain CERS 382.20 by conjugation, and transconjugants were selected at 30° C. on plates supplemented with chloramphenicol (Cm). Individual plasmid-containing colonies were picked and passaged twice at 42° C. in broth culture and then plated on pre-warmed Cm plates to select for integration of the plasmid at the inlB locus. Single colonies were picked from the high-temperature plates and passaged non-selectively in broth at 30° C. 5-10 times and plated for single colonies at 30° C. Clones containing the HCV NS5b-NS3 consensus sequence expression cassette were selected based on the following criteria: Streptomycin resistance, chloramphenicol sensitivity, PCR positive for HCV NS5b-NS3 sequences, PCR negative for pKSV7 vector sequences, and confirmation of genomic locus by PCR with a primer that anneals within the NS5b-encoding sequence insertion and the second primer outside of the 1.3 kb used to direct the homologous recombination.

Clones were screened for expression of the ActA-N100-HCV NS5B-NS3 fusion protein in Lm infected DC2.4 tissue culture cells by Western blot using an antibody directed against the mature N-terminus of ActA. The final clone (BH2064) was completely sequenced at the inlB locus, which confirmed the precise insertion of the expression cassette, and it was also tested for the induction of HCV-specific T cell responses and biodistribution in mouse models of infection.

The working ANZ-521 vaccine product comprises 1.5 mL of attenuated L. monocytogenes strain BH2064 at a nominal titer of 1×10¹⁰ cfu/mL in Dulbecco's phosphate buffered saline (DPBS) and 9% v/v glycerol. The product may be stored frozen at or below −60° C.

### Example 4

### Immunogenicity of Bacterially Expressed HCV Antigens

Recombinant L. monocytogenes has been shown to induce potent CD4+ and CD8+ T cell immunity to the encoded heterologous antigen in mice. The ability of Lm ΔactA/ΔinlB expressing HCV NS5b-N3 to induce NS5b- and NS3-specific T cell immunity was determined in various mouse strains following a single immunization. Initially a construct which contains an additional SL8 mouse T cell epitope at the C-terminus of the HCV antigen sequence was used to establish immunogenicity. This was later confirmed using ANZ 521, which lacks the SL8 epitope.

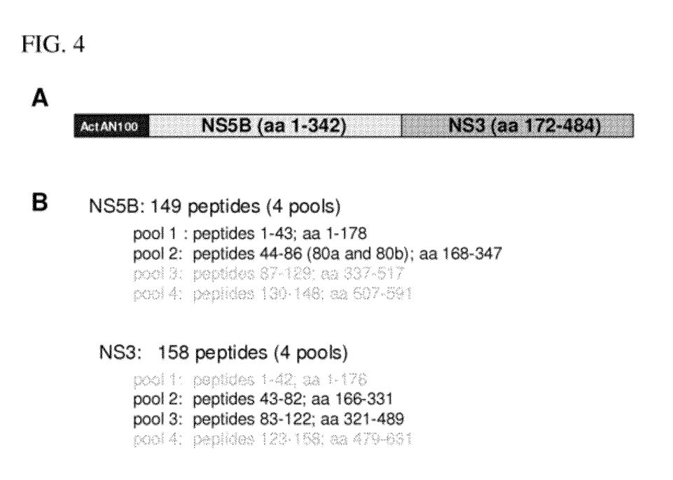

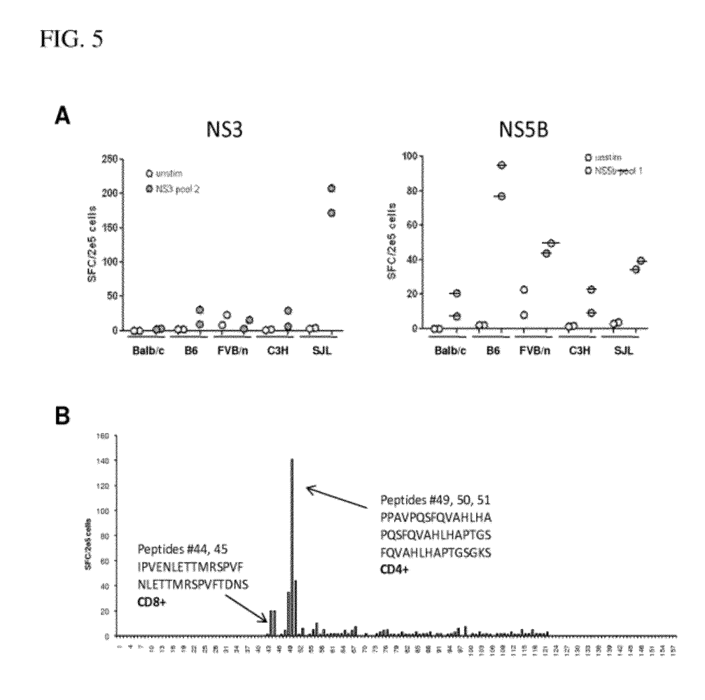

NS5b- and NS3-specific CD4+ and CD8+ T cell responses were determined by ELISPOT or intracellular cytokine analysis assays using peptide libraries comprising nested 15 amino acid peptides which overlap by 11 amino acids. The libraries span the complete sequence of HCV NS5b and NS3. Pools 1 and 2 of the NS5b peptide library cover the NS5b fragment; pools 2 and 3 of the NS3 peptide library cover the NS3 fragment expressed by ANZ-521 (FIG. 4). Initial experiments were conducted with the SL8 tag construct. This construct induced NS3-specific immunity in SJL mice and NS5b-specific immunity in all mouse strains evaluated: Balb/c, C57BL/6, FVB/n, C3H and SJL mice (FIG. 5A).

The NS3-specific T cell response in SJL located to two regions of the HCV NS3 protein: amino acids covered by peptides 44/45 and peptides 49 to 51 (FIG. 5B). These correspond to the following sequences: IPVENLETTMRSPVF (SEQ ID NO: 1); NLETTMRSPVFTDNS (SEQ ID NO: 2); PPAVPQSFQVAHLHA (SEQ ID NO: 3); PQSFQVAHLHAPTGS (SEQ ID NO: 4); and FQVAHLHAPTGSGKS (SEQ ID NO: 5). Subsequent experiments identified these regions as CD8+ and CD4+ T cell epitopes, respectively (data not shown).

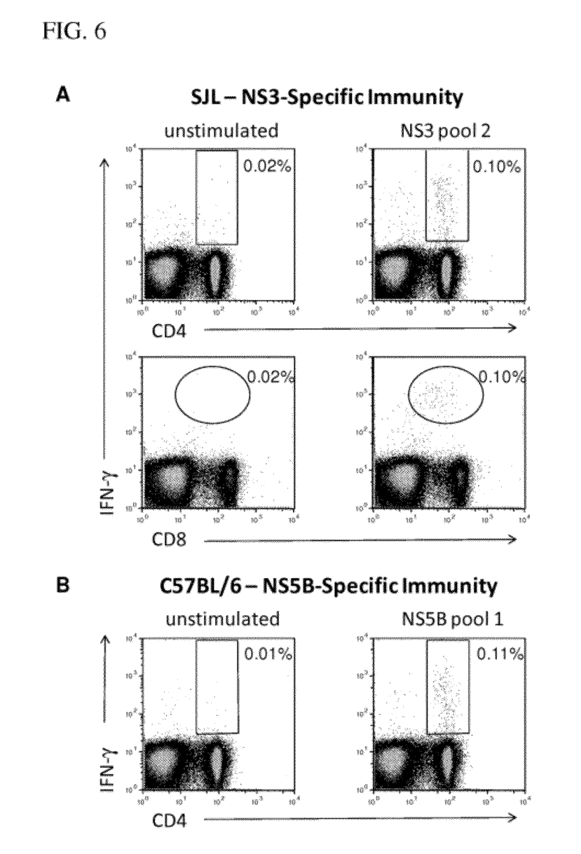

NS3- and NS5b-specific CD4+ and CD8+ T cell immunity was also demonstrated by intracellular cytokine analysis in mice following a single intravenous administration of ANZ-521 (FIG. 6). Splenocytes from immunized mice were stimulated for 5 hours with the relevant peptide in the presence of brefeldin A for intracellular cytokine staining Stimulated cells were surface stained for CD4 and CD8, then fixed and permeabilized using the cytofix/cytoperm kit (BD Biosciences, San Jose, Calif.). Cells were then stained for IFN-γ, TNF-α and/or IL-2. Samples were acquired using a FACSCanto flow cytometer (BD Biosciences). Data were gated to include exclusively CD4+ or CD8+ events, then the percentage of these cells expressing IFN-γ determined.

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

1-93. (canceled)

94. A method of inducing a T-cell response to hepatitis C virus (HCV) in a subject, said method comprising:
administering to said subject a composition comprising a bacterium which expresses one or more immunogenic HCV antigen polypeptides, the amino acid sequence of which comprise
(i) one or more full length HCV proteins selected from the group consisting of core, E1, E2, p7, NS2, NS3, NS4a, NS4b, NS5a, and NS5b;
(ii) one or more immunogenic amino acid sequences derived from one or more full length HCV proteins from (i); or
a combination of one or more full length HCV proteins of (i) and one or more amino acid sequences of (ii);
under conditions selected to induce said T cell response in said subject.

95. The method of claim 94 wherein said immunogenic HCV antigen polypeptide(s) comprise one or more amino acid sequences selected from the group consisting of full length NS3, full length NS5b, an amino acid sequence derived from NS3, and an amino acid sequence derived from NS5b.

96. The method of claim 94 wherein said immunogenic HCV antigen polypeptide(s) comprise one or more amino acid sequences selected from the group consisting of an amino acid sequence comprising at least 100 contiguous residues from NS3, and an amino acid sequence comprising at least 100 contiguous residues from NS5b.

97. The method of claim 94 wherein said immunogenic HCV antigen polypeptide(s) comprise one or more amino acid sequences selected from the group consisting of an amino acid sequence having at least 90% sequence identity to at least 100 contiguous residues from NS3, and an amino acid sequence having at least 90% sequence identity to at least 100 contiguous residues from NS5b.

98. The method of claim 94 wherein said immunogenic HCV antigen polypeptide(s) comprise one or more amino acid sequences selected from the group consisting of SEQ ID NOS: 1, 2, 3, 4, 5, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, and 83.

99. The method of claim 94, wherein the bacterium is Listeria monocytogenes comprising a nucleic acid sequence encoding said one or more immunogenic HCV antigen polypeptides integrated into the genome of said bacterium.

100. The method of claim 99, wherein the bacterium is an actA deletion mutant or an actA insertion mutant, an inlB deletion mutant or an inlB insertion mutant or a ΔactA/ΔinlB mutant comprising both an actA deletion or an actA insertion and an inlB deletion or an inlB insertion.

101. The method of claim 99, wherein a polynucleotide encoding one or more of said immunogenic HCV antigen polypeptide(s) has been integrated into a virulence gene of said bacterium, and the integration of the polynucleotide disrupts expression of the virulence gene or disrupts a coding sequence of the virulence gene.

102. The method of claim 101, wherein the virulence gene is actA or inlB.

103. The method of claim 99, wherein the bacterium is an attenuated Listeria monocytogenes.

104. The method of claim 103, wherein the bacterium is Lm ΔactA/ΔinlB.

105. The method of claim 99, wherein the bacterium further comprises a genetic mutation that attentuates the ability of the bacterium to repair nucleic acid.

106. The method of claim 105, wherein the genetic mutation is in one or more genes selected from phrB, uvrA, uvrB, uvrC, uvrD and recA.

107. The method of claim 103, wherein the bacterium is a Listeria monocytogenes prfA mutant, the genome of which encodes a prfA protein which is constitutively active.

108. The method of claim 99, wherein the bacterium is a killed but metabolically active Listeria monocytogenes.

109. The method of claim 108, wherein the bacterium is a Listeria monocytogenes prfA mutant, the genome of which encodes a prfA protein which is constitutively active.

110. The method of claim 99, wherein the nucleic acid sequence is codon optimized for expression by Listeria monocytogenes.

111. The method of claim 99, wherein said conditions selected to induce said T cell response in said subject comprise administering said Listeria monocytogenes by one or more routes of administration selected from the group consisting of orally, intramuscularly, intravenously, intradermally, and subcutaneously to said subject.

112. The method of claim 94, wherein said immunogenic HCV antigen polypeptide(s) are expressed as a fusion protein comprising an in frame ActA-N100 sequence selected from the group consisting of SEQ ID NO: 37, SEQ ID NO: 38, SEQ ID NO: 39, SEQ ID NO: 40, or an amino acid sequence having at least 90% sequence identity to said ActA-N100 sequence.

113. The method of claim 94, wherein said method comprises administering a Listeria monocytogenes expressing a fusion protein comprising:
(a) an ActA-N100 sequence selected from the group consisting of SEQ ID NO: 37, SEQ ID NO: 38, SEQ ID NO: 39, SEQ ID NO: 40 or an amino acid sequence having at least 90% sequence identity to said ActA-N100 sequence;
(b) an amino acid sequence comprising at least 100 contiguous residues from NS3 or an amino acid sequence having at least 90% sequence identity to an amino acid sequence comprising at least 100 contiguous residues from NS3; and
(c) an amino acid sequence comprising at least 100 contiguous residues from NS5b or an amino acid sequence having at least 90% sequence identity to an amino acid sequence comprising at least 100 contiguous residues from NS5b;
wherein said fusion protein is expressed from a nucleic acid sequence operably linked to a Listeria monocytogenes ActA promoter.

114. The method of claim 113, wherein said Listeria monocytogenes expresses a fusion protein comprising amino acids 1-342 of NS5b having the sequence of SEQ ID NO: 18 or SEQ ID NO: 19, or a mutated derivative thereof wherein said mutation inactivates the RNA polymerase activity of NS5b; and amino acids 172-484 of NS3 having the sequence of SEQ ID NO: 13 or SEQ ID NO: 14, or a mutated derivative thereof wherein said mutation inactivates the helicase activity of NS3.

115. The method of claim 94, wherein said immunogenic HCV antigen polypeptide(s) comprise one or more contiguous HCV amino acid sequences having no region of hydrophobicity that exceeds the peak hydrophobicity of Listeria ActA-N100.

116. A composition comprising:
a bacterium which comprises a nucleic acid molecule, the sequence of which encodes one or more immunogenic HCV antigen polypeptides, the amino acid sequence of which comprise
(i) one or more full length HCV proteins selected from the group consisting of core, E1, E2, p7, NS2, NS3, NS4a, NS4b, NS5a, and NS5b;
(ii) one or more immunogenic amino acid sequences derived from one or more full length HCV proteins from (i); or
a combination of one or more full length HCV proteins of (i) and one or more amino acid sequences of (ii).

117. A method of HCV prophylaxis or of treating a chronic HCV infection in a subject, said method comprising:
administering to said subject a composition comprising a bacterium which expresses one or more immunogenic HCV antigen polypeptides, the amino acid sequence of which comprise
(i) one or more full length HCV proteins selected from the group consisting of core, E1, E2, p7, NS2, NS3, NS4a, NS4b, NS5a, and NS5b;
(ii) one or more immunogenic amino acid sequences derived from one or more full length HCV proteins from (i); or
a combination of one or more full length HCV proteins of (i) and one or more amino acid sequences of (ii);
under conditions selected to induce said T cell response in said subject.
